(19) United States
(12) Patent Application Publication
(10) Pub. No.: US 2006/0014734 A1
(43) Pub. Date: Jan. 19, 2006

(54) Immediate release pharmaceutical formulation

(76) Inventors: Susanna Abrahmsen Alami, Molndal (SE); Tord Inghardt, Molndal (SE); Anders Magnusson, Molndal (SE); Carl-Gustaf Sigfridsson, Molndal (SE); Mikael Thune, Molndal (SE)

Correspondence Address: FISH & NEAVE IP GROUP;ROPES & GRAY LLP, ONE INTERNATIONAL PLACE, BOSTON, MA, 02110-2624, US

(21) Appl. No.: 10/516,423

(22) PCT Filed: May 27, 2003
(86) PCT No.: PCT/SE03/00857

§ 371 Date: Jul. 25, 2005

(30) Foreign Application Priority Data
May 31, 2002 (SE) 0201658-2

(51) Int. Cl. A61K 31/397; A61K 9/20

(52) U.S. Cl. 514/210.17; 424/464

(57) ABSTRACT

According to the present invention there is provided an immediate release pharmaceutical formulation comprising, as active ingredient, a compound of formula (I): 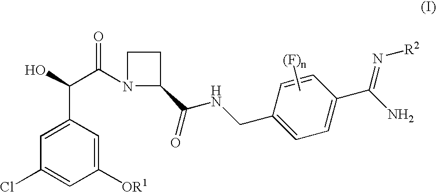 wherein
R¹ represents C₁₋₂ alkyl substituted by one or more fluoro substituents;
R² represents hydrogen, hydroxy, methoxy or ethoxy; and n represents 0, 1 or 2; or a pharmaceutically acceptable salt thereof; and a pharmaceutically acceptable diluent or carrier; provided that when the active ingredient is other than in the form of a salt the formulation does not solely contain: a solution of one active ingredient and water, a solution of one active ingredient and dimethylsulphoxide; or a solution of one active ingredient in a mixture of ethanol:PEG 660 12-hydroxy stearate:water 5:5:90; such formulations being of use for the treatment of a cardiovascular disorder.

This application is a national stage filing under 35 U.S.C. 371 of International Application No. PCT/SE03/00857, filed May 27, 2003, which claims priority from Sweeden Application No. 0201658-2, filed May 31, 2002, the specification of which is incorporated by reference herein. International Application No. PCT/SE03/00857 was published under PCT Article 21(2) in English.

This invention relates to a novel immediate release pharmaceutical formulation that provides for the delivery of particular pharmaceuticals, to the manufacture of such a formulation, and to the use of such a formulation in the treatment or prevention of thrombosis.

It is often desirable to formulate pharmaceutically active compounds for immediate release following oral and/or parenteral administration with a view to providing a sufficient concentration of drug in plasma within the time-frame required to give rise to a desired therapeutic response.

Immediate release may be particularly desirable in cases where, for example, a rapid therapeutic response is required (for example in the treatment of acute problems), or, in the case of parenteral administration, when peroral delivery to the gastrointestinal tract is incapable of providing sufficient systemic uptake within the required time-frame.

In the case of the treatment or prophylaxis of thrombosis, immediate release formulations may be necessary to ensure that a sufficient amount of drug is provided in plasma within a relatively short period of time to enable quick onset of action. Immediate release formulations are also typically simpler to develop than modified release formulations, and may also provide more flexibility in relation to the variation of doses that are to be administered to patients. Immediate release formulations are superior when multiple doses are not required and where it is not necessary to keep the plasma concentration at a constant level for an extended time.

International Patent Application No. PCT/SE01/02657 (WO 02/44145, earliest priority date 1 Dec. 2000, filed 30 Nov. 2001, published 6 Jun. 2002) discloses a number of compounds that are, or are metabolised to compounds which are, competitive inhibitors of trypsin-like proteases, such as thrombin. The following three compounds are amongst those that are specifically disclosed:
(a) Ph(3-Cl)(5-OCHF₂)—(R)CH(OH)C(O)—(S)Aze-Pab(OMe): 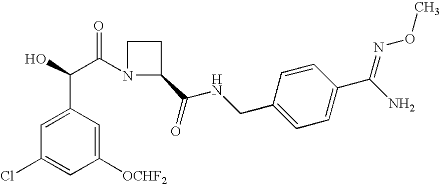
   which compound is referred to hereinafter as Compound A;
(b) Ph(3-Cl)(5-OCHF₂)—(R)CH(OH)C(O)—(S)Aze-Pab(2,6-diF)(OMe): 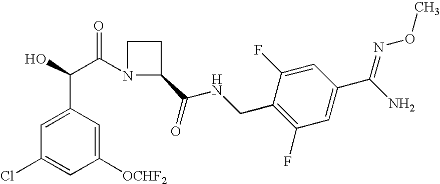
   which compound is referred to hereinafter as Compound B; and
(c) Ph(3-Cl)(5-OCH₂CH₂F)—(R)CH(OH)C(O)—(S)Aze-Pab(OMe): 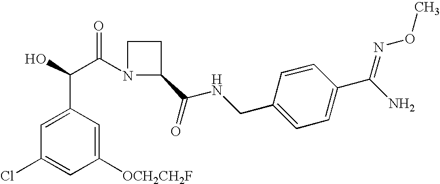
   which compound is referred to hereinafter as Compound C.

The methoxyamidine Compounds A, B and C are metabolised following oral and/or parenteral administration to the corresponding free amidine compounds, which latter compounds have been found to be potent inhibitors of thrombin. Thus:
Compound A is metabolized to Ph(3-Cl)(5-OCHF₂)—(R)CH(OH)C(O)—(S)Aze-Pab (which compound is referred to hereinafter as Compound D) via a prodrug intermediate Ph(3-Cl)(5-OCHF₂)—(R)CH(OH)C(O)—(S)Aze-Pab(OH) (which compound is referred to hereinafter as Compound G);
Compound B is metabolized to Ph(3-Cl)(5-OCHF₂)—(R)CH(OH)C(O)—(S)Aze-Pab(2,6-diF) (which compound is referred to hereinafter as Compound E) via a prodrug intermediate Ph(3-Cl)(5-OCHF₂)—(R)CH(OH)C(O)—(S)Aze-Pab(2,6-diF)(OH) (which compound is referred to hereinafter as Compound H); and,
Compound C is metabolized to Ph(3-Cl)(5-OCH₂CH₂F)—(R)CH(OH)C(O)—(S)Aze-Pab (which compound is referred to hereinafter as Compound F) via a prodrug intermediate Ph(3-Cl)(5-OCH₂CH₂F)—(R)CH(OH)C(O)—(S)Aze-Pab(OH) (which compound is referred to hereinafter as Compound J).

Processes for the synthesis of Compounds A, B, C, D, E, F, G and J are described in Examples 12, 40, 22, 3, 39, 21, 2 and 31 (respectively) of international patent application No. PCT/SE01/02657. An immediate release formulation of these compounds, or their metabolites has yet to be described in the literature. We have found that the compounds of formula (I) and their salts can be formulated as immediate release pharmaceutical formulations which are easy to administer, for example by oral or parenteral administration.

According to a first aspect of the invention, there is provided an immediate release pharmaceutical formulation comprising, as active ingredient, a compound of formula (I): 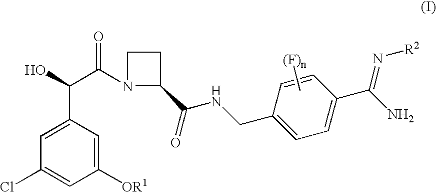
wherein
R¹ represents C₁₋₂ alkyl substituted by one or more fluoro substituents;
R² represents hydrogen, hydroxy, methoxy or ethoxy; and
n represents 0, 1 or 2;
   or a pharmaceutically acceptable salt thereof; and a pharmaceutically acceptable diluent or carrier; provided that the formulation does not solely contain:
   a solution of one active ingredient and water;
   a solution of one active ingredient and dimethylsulphoxide; or,
   a solution of one active ingredient in a mixture of ethanol:PEG 660 12-hydroxy stearate:water 5:5:90;
      which formulations are referred to hereinafter as “the formulations of the invention”.

PEG 660 12-hydroxy stearate is a non-ionic surfactant and is better known as Solutol K™.

According to a second aspect of the present invention there is provided Compound H, Ph(3-Cl)(5-OCHF₂)—(R)CH(OH)C(O)—(S)Aze-Pab(2,6-diF)(OH) which can be prepared by methods similar to those described below for the preparation of Compounds G and J.

The compounds of formula (I), or a pharmaceutically acceptable salt thereof, may be in the form of a solvate, a hydrate, a mixed solvate/hydrate or, preferably, an ansolvate, such as an anhydrate. Solvates may be of one or more organic solvents, such as lower (for example C₁₋₄) alkyl alcohols (for example methanol, ethanol or iso-propanol), ketones (such as acetone), esters (such as ethyl acetate) or mixtures thereof.

In one particular aspect of the invention R¹ is CHF₂ or CH₂CH₂F.

The variable n is preferably 0 or 2.

More preferred compounds of formula (I) include those in which n represents 0, or those in which n represents 2, so providing two fluoro atoms located at the 2- and 6-positions (that is the two ortho-positions relative to the point of attachment of the benzene ring to the —NH—CH₂— group).

The compound of formula (I) is especially Compound A, Compound B or Compound C.

Preferred salts of the compounds of formula (I) are acid addition salts. Acid addition salts include inorganic acid addition salts, such as those of sulphuric acid, nitric acid, phosphoric acid and hydrohalic acids, such as hydrobromic acid and hydrochloric acid. More preferred acid addition salts include those of organic acids, such as those of dimethylphosphoric acid; saccharinic acid; cyclohexylsulfamic acid; those of carboxylic acids (such as maleic acid, fumaric acid, aspartic acid, succinic acid, malonic acid, acetic acid, benzoic acid, terephthalic acid, hippuric acid, 1-hydroxy-2-naphthoic acid, pamoic acid, hydroxybenzoic acid and the like); those of hydroxy acids (such as salicylic acid, tartaric acid, citric acid, malic acid (including L-(−)-malic acid and, D,L-malic acid), gluconic acid (including D-gluconic acid), glycolic acid, ascorbic acid, lactic acid and the like); those of amino acids (such as glutamic acid (including D-glutamic, L-glutamic, and D,L-glutamic, acids), arginine (including L-arginine), lysine (including L-lysine and L-lysine hydrochloride), glycine and the like); and, particularly, those of sulfonic acids, (such as 1,2-ethanedisulfonic acid, camphorsulfonic acids (including 1S-(+)-10-camphorsulfonic acid and (+/−)-camphorsulfonic acids), ethanesulfonic acid, a propanesulfonic acid (including n-propanesulfonic acid), a butanesulfonic acid, a pentanesulfonic acid, a toluenesulfonic acid, methanesulfonic acid, p-xylenesulfonic acid, 2-mesitylenesulfonic acid, naphthalenesulfonic acids (including 1,5-naphthalenesulfonic acid and naphthalenesulfonic acid), benzenesulfonic acid, hydroxybenzenesulfonic acids, 2-hydroxyethanesulfonic acid, 3-hydroxyethanesulfonic acid and the like).

Particularly preferred salts include those of C₁₋₆ (for example C₁₋₄) alkanesulfonic acids, such as ethanesulfonic acid (esylate) and propanesulfonic acid (for example n-propanesulfonic acid) and optionally substituted (for example with one or more C₁₋₂ alkyl groups) arylsulfonic acids, such as benzenesulfonic acid (besylate) and naphthalenedisulfonic acid.

Suitable stoichiometric ratios of acid to free base are in the range 0.25:1.5 to 3.0:1, such as 0.45:1.25 to 1.25:1, including 0.50:1 to 1:1.

According to a further aspect of the invention there is provided formulation comprising a compound of formula (I) in substantially crystalline form.

Although we have found that it is possible to produce compounds of the invention in forms which are greater than 80% crystalline, by “substantially crystalline” we include greater than 20%, preferably greater than 30%, and more preferably greater than 40% (e.g. greater than any of 50, 60, 70, 80 or 90%) crystalline.

According to a further aspect of the invention there is also provided a compound of the invention in partially crystalline form. By “partially crystalline” we include 5% or between 5% and 20% crystalline.

The degree (%) of crystallinity may be determined by the skilled person using X-ray powder diffraction (XRPD). Other techniques, such as solid state NMR, FT-IR, Raman spectroscopy, differential scanning calorimetry (DSC) and microcalorimetry, may also be used.

Preferred compounds of formula (I) that may be prepared in crystalline form include salts of C₁₋₆ (for example C₂₋₆, such as C₂₋₄) alkanesulfonic acids, such as ethanesulfonic acid, propanesulfonic acid (for example n-propanesufonic acid) and optionally substituted arylsulfonic acids, such as benzenesulfonic acid and naphthalenedisulfonic acid.

The term “immediate release” pharmaceutical formulation includes any formulation in which the rate of release of drug from the formulation and/or the absorption of drug, is neither appreciably, nor intentionally, retarded by galenic manipulations. In the present case, immediate release may be provided for by way of an appropriate pharmaceutically acceptable diluent or carrier, which diluent or carrier does not prolong, to an appreciable extent, the rate of drug release and/or absorption. Thus, the term excludes formulations which are adapted to provide for “modified”, “controlled”, “sustained”, “prolonged”, “extended” or “delayed” release of drug.

In this context, the term “release” includes the provision (or presentation) of drug from the formulation to the gastrointestinal tract, to body tissues and/or into systemic circulation. For gastrointestinal tract release, the release is under pH conditions such as pH=1 to 3, especially at, or about, pH=1. In one aspect of the invention a formulation as described herein with a compound of formula (I), or an acid addition salt thereof, in crystalline form releases drug under a range of pH conditions. In another aspect of the invention a formulation as described herein with a compound of formula (I), or an acid addition salt thereof, releases drug under pH conditions such as pH=1 to 3, especially at, or about, pH=1. Thus, formulations of the invention may release at least 70% (preferably 80%) of active ingredient within 4 hours, such as within 3 hours, preferably 2 hours, more preferably within 1.5 hours, and especially within an hour (such as within 30 minutes), of administration, whether this be oral or parenteral.

The formulations of the invention may be formulated in accordance with a variety of known techniques, for example as described by M. E. Aulton in “Pharmaceutics: The Science of Dosage Form Design” (1988) (Churchill Livingstone), the relevant disclosures in which document are hereby incorporated by reference.

Formulations of the invention may be, or may be adapted in accordance with standard techniques to be, suitable for peroral administration, for example in the form of an immediate release tablet, an immediate release capsule or as a liquid dosage form, comprising active ingredient. These formulation types are well known to the skilled person and may be prepared in accordance with techniques known in the art.

Suitable diluents/carriers (which may also be termed “fillers”) for use in peroral formulations of the invention, for example those in the form of immediate release tablets, include monobasic calcium phosphate, dibasic calcium phosphate (including dibasic calcium phosphate dihydrate and dibasic calcium phosphate anhydrate), tribasic calcium phosphate, lactose, microcrystalline cellulose, silicified microcrystalline cellulose, mannitol, sorbitol, starch (such as maize, potato or rice), glucose, calcium lactate, calcium carbonate and the like. Preferred diluents/carriers include dibasic calcium phosphate and microcrystalline cellulose, which may be used alone or in combination with another diluent/carrier such as mannitol.

A formulation of the invention in the form of an immediate release tablet may comprise one or more excipients to improve the physical and/or chemical properties of the final composition, and/or to facilitate the process of manufacture. Such excipients are conventional in the formulation of immediate release formulations for peroral drug delivery, and include one or more of the following: one or more lubricants (such as magnesium stearate, stearic acid, calcium stearate, stearyl alcohol or, preferably, sodium stearyl fumarate); a glidant (such as talc or a colloidal silica); one or more binders (such as polyvinylpyrrolidone, microcrystalline cellulose, a polyethylene glycol (PEG), a polyethylene oxide, a hydroxypropylmethylcellulose (HPMC) of a low molecular weight, a methylcellulose (MC) of a low molecular weight, a hydroxypropylcellulose (HPC) of a low molecular weight, a hydroxyethylcellulose (HEC) of a low molecular weight, a starch (such as maize, potato or rice) or a sodium carboxymethyl cellulose of a low molecular weight; (preferred binders are polyvinylpyrrolidone or a HPMC of a low molecular weight); one or more pH controlling agents (such as an organic acid (for example citric acid) or an alkali metal (for example sodium) salt thereof, an oxide of magnesium, an alkali or alkaline earth metal (for example sodium, calcium or potassium) sulphate, metabisulphate, propionate or sorbate); one or more disintegrant (for example sodium starch glycollate, a crosslinked polyvinylpyrrolidone, a crosslinked sodium carboxymethyl cellulose, a starch (such as maize, potato or rice) or an alginate); a colourant, a flavouring, a tonicity-modifying agent, a coating agent or a preservative.

It will be appreciated that some of the above mentioned excipients which may be present in a final immediate release oral (for example tablet) formulation of the invention may have more than one of the above-stated functions.

In a further aspect of the invention a liquid formulation of the invention is adapted to be suitable for oral administration.

Suitable liquid formulations that are to be administered orally include those in which a compound of formula (I) especially Compound A, Compound B or Compound C, or a pharmaceutically acceptable salt thereof is presented together with an aqueous carrier, such as water. It will be noted however, that certain specific formulations are not claimed (see particular aspects and the claims).

A formulation of the present invention comprising an aqueous carrier may further comprise one or more excipients, such as an antimicrobial preservative; a tonicity modifier (for example sodium chloride, mannitol or glucose); a pH adjusting agent (for example a common inorganic acid or base, including hydrochloric acid or sodium hydroxide); a pH controlling agents (that is, a buffer; for example tartaric acid, acetic acid or citric acid); a surfactant (for example Sodiun dodecyl sulphate (SDS) or Solutol™); a solubiliser which serves to help solubilise the active ingredient (for example ethanol, a polyethylene glycol or hydroxypropyl-β-cyclodextrin or polyvinyl chloride (PVP)); or an antioxidant.

Liquid oral formulations may be in the form of suspensions of active ingredient in association with an aqueous solvent or, more preferably aqueous solutions (that is, solutions of active compound including water as a solvent). In this context, the term “aqueous solution” includes formulations in which at least 99% of active ingredient is in solution at above 5° C. and atmospheric pressure, and the term “suspension” means that more than 1% of active ingredient is not in solution under such conditions. Typical dispersion agents for suspensions are hydroxypropyl methylcellulose, AOT (dioctylsulfosuccinate), PVP and SDS. Other alternatives may be possible.

In another aspect the present invention provides a liquid oral formulation comprising a compound of formula (I), or a pharmaceutically acceptable salt thereof, water and at least one additional agent. The additional agents include:
i. polyethylene glycol (PEG) and optionally also ethanol and/or tartaric acid and/or citric acid and/or hydrochloric acid; or
ii. sodium chloride (which will be dissolved in the formulation), and optionally also ethanol; or
iii. hydrochloric acid and/or sodium hydroxide to bring the pH to a suitable value (preferably in the range 3-8 for a compound of formula (I) wherein R² is methoxy or ethoxy, such as Compound A, B or C); or
iv. DMA (dimethyl acetamide) and optionally also a medium chain triglyceride (such as miglyol); or
v. a β-cyclodextrin (such as hydroxypropyl-β-cyclodextrin);
vi. a tonicity modifier such as sodium chloride and/or mannitol.

In a further aspect the present invention provides an oral solution comprising a compound of formula (I), or a pharmaceutically acceptable salt thereof, (preferably Compound A, B or C) water and at least one additional agents as recited in (i) to (vi) above.

In another aspect the invention provides an aqueous formulation of a compound of formula (I) (such as Compound A, B or C) comprising a solubilising agent such as a polyethylene glycol, β-cyclodextrin (such as hydroxypropyl-β-cyclodextrin), sorbitol or ethanol.

In a further aspect the present invention provides an oral solution formulation comprising a compound of formula (I) and ethanol. This formulation can further comprise a medium chain triglyceride (such as miglyol).

In a still further aspect the present invention provides an oral solution formulation comprising a compound of formula (I) and DMA. This formulation can further comprise a medium chain triglyceride (such as miglyol).

In another aspect the compound of formula (I) is crystalline (especially a salt of Compound A; preferably a C₁₋₆ (for example C₂₋₆, such as C₂₋₄) alkanesulfonic acid salt, such as ethanesulfonic acid, propanesulfonic acid (for example n-propanesufonic acid) or an optionally substituted arylsulfonic acid salt, such as benzenesulfonic acid or naphthalenedisulfonic acid salt).

A particular liquid immediate release oral pharmaceutical formulation as claimed in claim 1 is provided wherein the active ingredient is:
Ph(3-Cl)(5-OCHF₂)—(R)CH(OH)C(O)—(S)Aze-Pab(OMe),
Ph(3-Cl)(5-OCHF₂)—(R)CH(OH)C(O)—(S)Aze-Pab(2,6-diF)(OMe),
Ph(3-Cl)(5-OCH₂CH₂F)—(R)CH(OH)C(O)—(S)Aze-Pab(OMe),
   or a pharmaceutically acceptable salt thereof.

A further particular liquid immediate release oral pharmaceutical formulation as claimed in claim 1 is provided wherein the active ingredient is: Ph(3-Cl)(5-OCHF₂)—(R)CH(OH)C(O)—(S)Aze-Pab(OMe) or a C₁₋₄ alkanesulfonic acid or an optionally substituted arylsulfonic acid salt thereof

A yet further particular liquid immediate release oral pharmaceutical formulation as claimed in claim 1 is provided wherein the active ingredient is: Ph(3-Cl)(5-OCHF₂)—(R)CH(OH)C(O)—(S)Aze-Pab(2,6-diF)(OMe) or an optionally substituted arylsulfonic acid salt thereof (such as the naphthalene-1,5-disulphonic acid salt).

It will be noted however, that certain specific formulations are not claimed (see particular aspects and the claims).

In a further aspect of the invention a formulation of the invention is adapted to be suitable for parenteral administration. The term “parenteral” includes any mode of administration that does not comprise peroral administration to the gastrointestinal tract and includes administration subcutaneously, intravenously, intraarterially, transdermally, intranasally, intrabuccally, intracutaneously, intramuscularly, intralipomateously, intraperitoneally, rectally, sublingually, topically, by inhalation, or by any other parenteral route.

Suitable formulations of the invention that are to be administered parenterally include those in which a compound of formula (I) or a pharmaceutically acceptable salt thereof is presented together with an aqueous carrier, such as water.

A formulation of the present invention comprising an aqueous carrier may further comprise one or more excipients, such as an antimicrobial preservative; a tonicity modifier (for example sodium chloride, mannitol or glucose); a pH adjusting agent (for example a common inorganic acid or base, including hydrochloric acid or sodium hydroxide); a pH controlling agents (that is, a buffer; for example tartaric acid, acetic acid or citric acid); a surfactant (for example sodium dodecyl sulphate (SDS) or Solutol™); a solubiliser which serves to help solubilise the active ingredient (for example ethanol, a polyethylene glycol or hydroxypropyl-β-cyclodextrin or polyvinyl chloride (PVP)); or an antioxidant.

Parenteral formulations may be in the form of suspensions of active ingredient in association with an aqueous solvent or, more preferably aqueous solutions (that is, solutions of active compound including water as a solvent). In this context, the term “aqueous solution” includes formulations in which at least 99% of active ingredient is in solution at above 5° C. and atmospheric pressure, and the term “suspension” means that more than 1% of active ingredient is not in solution under such conditions. Typical dispersion agents for suspensions are hydroxypropyl methylcellulose, AOT, PVP and SDS, but other alternatives are possible.

The number of excipients employed in the peroral and parenteral formulations of the invention depends upon many factors, such as the nature and amount of active ingredient present, and the amount of diluent/carrier (aqueous solvent or otherwise) that is included.

In another aspect the present invention provides a parenteral formulation comprising a compound of formula (I), or a pharmaceutically acceptable salt thereof, water and at least one additional agents. The additional agents include:
i. polyethylene glycol (PEG) and optionally also ethanol and/or tartaric acid and/or hydrochloric acid; or
ii. sodium chloride (which will be dissolved in the formulation), and optionally also ethanol; or
iii. hydrochloric acid and/or sodium hydroxide to bring the pH to a suitable value (preferably in the range 3-8 for a compound of formula (I) wherein R² is hydrogen, such as Compound D, E or F; or preferably in the range 3.5-8 for a compound of formula (I) wherein R² is methoxy or ethoxy, such as Compound A, B or C); or
iv. DMA (dimethyl acetamide) and optionally also a medium chain triglyceride (such as miglyol); or
v. a β-cyclodextrin (such as hydroxypropyl-β-cyclodextrin);
vi. a tonicity modifier such as sodium chloride and/or mannitol.

In a further aspect the present invention provides an injectable solution comprising a compound of formula (I), or a pharmaceutically acceptable salt thereof, (preferably Compound D, E or F) water and at least one additional agents as recited in (i) to (vi) above.

In another aspect the invention provides an aqueous formulation of a compound of formula (I) (such as Compound D, E or F) comprising a solubilising agent such as a polyethylene glycol, β-cyclodextrin (such as hydroxypropyl-β-cyclodextrin), sorbitol or ethanol.

In a further aspect the present invention provides a parenteral formulation comprising a compound of formula (I) and ethanol. This formulation can further comprise a medium chain triglyceride (such as miglyol).

In a still further aspect the present invention provides a parenteral formulation comprising a compound of formula (I) and DMA. This formulation can further comprise a medium chain triglyceride (such as miglyol).

In another aspect the compound of formula (I) is crystalline (especially a salt of Compound A; preferably a C₁₋₆ (for example C₂₋₆, such as C₂₋₄) alkanesulfonic acid salt, such as ethanesulfonic acid, propanesulfonic acid (for example n-propanesufonic acid) or an optionally substituted arylsulfonic acid salt, such as benzenesulfonic acid salt).

In yet another aspect the formulation of the present invention is in a solid dosage form wherein R² is hydroxy, methoxy or ethoxy (preferably methoxy) (the compound of formula (I) is especially Compound A, Compound B or Compound C).

In yet another aspect the present invention provides a parenteral formulation (especially a water-based, injectable solution) comprising a compound of formula (I) in free base form.

In a further aspect the present invention provides a parenteral formulation comprising a compound of formula (I) in free base form wherein R² is hydrogen.

In a still further aspect the present invention provides a solid formulation comprising microcrystalline cellulose and polyvinyl pyrrolidone (PVP); or comprising microcrystalline cellulose and sodium starch glycollate.

Formulations of the invention, such as parenteral formulations, comprising salts may be prepared by addition of diluent/carrier to the appropriate pre-prepared salt.

Compositions including active ingredient may also be provided in solid form suitable for use in the preparation of a formulation of the invention (for example a solution, such as an aqueous solution, for example for parenteral adminstration) ex tempore. Such compositions may be in the form of a solid comprising active ingredient, optionally in the presence of one or more further excipients as hereinbefore defined and, optionally, up to 10% (w/w) of diluent and/or carrier as hereinbefore defined, which compositions are hereinafter referred to as “the solid compositions of the invention”.

Solid compositions of the invention may be made by removal of diluent/carrier (for example solvent) from a formulation of the invention, or a concentrated formulation of the invention, which may for example be in the form of a solution, such as an aqueous solution.

In another aspect the present invention provides an orally administerable, immediate release formulation comprising a compound of formula (I), or a salt thereof, a carrier (such as microcrystalline cellulose), a disintegrant (such as sodium starch glycollate), a binder (such as polyvinyl pyrrolidone) and a lubricant (such as sodium stearyl fumarate). Such a formulation may also comprise an additional carrier (or filler) such as mannitol.

Formulations of the invention that are in the form of immediate release tablets may be prepared by bringing active ingredient into association with diluent/carrier using standard techniques, and using standard equipment, known to the skilled person, including wet or dry granulation, direct compression/compaction, drying, milling, mixing, tableting and coating, as well as combinations of these processes, for example as described hereinafter. In one aspect of the invention, acid addition salts of compounds of formula (I) in crystalline form are formulated in tablets.

There is thus provided a process for the formation of a solid composition suitable for use in the preparation of a formulation of the invention (for example a solution, such as an aqueous solution) ex tempore, which process comprises removal of diluent/carrier (for example solvent) from a formulation of the invention, or a concentrated formulation of the invention.

Solvent may be removed by way of a variety of techniques known to those skilled in the art, for example evaporation (under reduced pressure or otherwise), freeze-drying, or any solvent removal (drying) process that removes solvent (such as water) while maintaining the integrity of the active ingredient. An example of drying is freeze-drying.

Thus according to a further aspect of the invention there is provided a freeze-dried (lyophilised) solid composition of the invention.

In the preparation of solid compositions of the invention, the skilled person will appreciate that appropriate additional excipients may be added at a suitable stage prior to removal of diluent/carrier. For example, in the case of aqueous solutions, pH may be controlled and/or adjusted as hereinbefore described. Furthermore, an appropriate additional excipient may be added with a view to aiding the formation of a solid composition of the invention during the process of diluent/carrier removal (for example mannitol, sucrose, glucose, mannose or trehalose).

A solid composition of a compound of formula (I) or a salt thereof, thus includes a composition in which the solvent (for example water) content, other than a solvent of crystallization, is no more than 10%, such as less than 2% unbound solvent, such as water.

Formulations of the invention may be sterilised, for example by sterile filtration or autoclavation, and/or filled into primary packages, such as vials, cartridges and pre-filled syringes. Such processing steps may also take place prior to drying to form a solid composition of the invention.

Before administration, the dried solid composition may be reconstituted and/or diluted in, for instance, water, physiological saline, glucose solution or any other suitable solution.

The amount of diluent/carrier in an oral (for example immediate release tablet) formulation of the invention depends upon many factors, such as the nature and amount of the active ingredient that is employed and the nature, and amounts, of any other constituents (for example further excipients) that are present in the formulation, but is typically up to 40% (w/w), preferably up to 30%, more preferably up to 20%, and particularly up to 10% (w/w) of the final composition. The amount of additional excipients in such an oral formulation of the invention also depends upon factors, such as the nature and amount of the active ingredient that is employed, as well as the nature, and amounts, of any other constituents (for example diluents/carriers and/or other further excipients) that are present in the formulation, but, for lubricants and glidants is typically up to 5% (w/w), and for binders and disintegrants is typically up to 10% (w/w) of the final composition.

The formulations of the invention are administered to mammalian patients (including humans), and, for compounds of formula (I) wherein R² is not hydrogen, are thereafter metabolised in the body to form compounds of formula (I) wherein R² is hydrogen that are pharmacologically active.

According to a further aspect of the invention there is thus provided a formulation of the invention for use as a pharmaceutical.

In particular, the compounds of formula (I) are, or are metabolised following administration to form, potent inhibitors of thrombin, for example as may be demonstrated in the tests described in inter alia international patent application No. PCT/SE01/02657, as well as international patent applications WO 02/14270, WO 01/87879 and WO 00/42059, the relevant disclosures in which documents are hereby incorporated by reference.

By “prodrug of a thrombin inhibitor”, we include compounds that are metabolised following administration and form a thrombin inhibitor, in an experimentally-detectable amount, following administration.

By “active ingredient” and “active substance” we mean the pharmaceutical agent (covering thrombin inhibitor and prodrugs thereof) present in the formulation.

The formulations of the invention are thus expected to be useful in those conditions where inhibition of thrombin is required, and/or conditions where anticoagulant therapy is indicated, including the following:

The treatment and/or prophylaxis of thrombosis and hypercoagulability in blood and/or tissues of animals including man. It is known that hypercoagulability may lead to thrombo-embolic diseases. Conditions associated with hypercoagulability and thrombo-embolic diseases which may be mentioned include inherited or acquired activated protein C resistance, such as the factor V-mutation (factor V Leiden), and inherited or acquired deficiencies in antithrombin III, protein C, protein S, heparin cofactor II. Other conditions known to be associated with hypercoagulability and thrombo-embolic disease include circulating antiphospholipid antibodies (Lupus anticoagulant), homocysteinemi, heparin induced thrombocytopenia and defects in fibrinolysis, as well as coagulation syndromes (for example disseminated intravascular coagulation (DIC)) and vascular injury in general (for example due to surgery).

The treatment of conditions where there is an undesirable excess of thrombin without signs of hypercoagulability, for example in neurodegenerative diseases such as Alzheimer's disease.

Particular disease states which may be mentioned include the therapeutic and/or prophylactic treatment of venous thrombosis (for example DVT) and pulmonary embolism, arterial thrombosis (e.g. in myocardial infarction, unstable angina, thrombosis-based stroke and peripheral arterial thrombosis), and systemic embolism usually from the atrium during atrial fibrillation (for example non-valvular atrial fibrillation) or from the left ventricle after transmural myocardial infarction, or caused by congestive heart failure; prophylaxis of re-occlusion (that is thrombosis) after thrombolysis, percutaneous trans-luminal angioplasty (PTA) and coronary bypass operations; the prevention of re-thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease and the formation of atherosclerotic plaques, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

The formulation of the present invention may also comprise any antithrombotic agent(s) with a different mechanism of action to that of the compounds of formula (I), such as one or more of the following: the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P₂T) antagonists; and inhibitors of carboxypeptidase U (CPU).

Compounds of formula (I) that inhibit trypsin and/or thrombin may also be useful in the treatment of pancreatitis.

The formulations of the invention are thus indicated both in the therapeutic and/or prophylactic treatment of these conditions.

According to a further aspect of the present invention, there is provided a method of treatment of a condition where inhibition of thrombin is required which method comprises administration of a therapeutically effective amount of a formulation of the invention to a person suffering from, or susceptible to, such a condition.

In a still further aspect the present invention provides a formulation of the invention in the manufacture of a medicament for use in the treatment of thrombosis.

According to a further aspect of the invention, there is provided a method of treatment of thrombosis which method comprises administration of a formulation of the invention to a person suffering from, or susceptible to, such a condition.

For the avoidance of doubt, by “treatment” we include the therapeutic treatment, as well as the prophylaxis, of a condition.

Suitable amounts of active ingredient in formulations (oral or parenteral), concentrated formulations, and solid compositions, of the invention depend upon many factors, such as the nature of that ingredient (free base/salt etc), the dose that is required in an oral formulation or in a final “ready to use” parenteral formulation that is, or is to be, prepared, and the nature, and amounts, of other constituents of the formulation. However, a typical daily dose of a compound of formula (I), or a pharmaceutically acceptable salt thereof, is in the range 0.001-100 mg/kg body weight at peroral administration and 0.001-50 mg/kg body weight at parenteral administration, excluding the weight of any acid counter-ion, irrespective of the number of individual doses that are administered during the course of that day. In the case of an immediate release parenteral formulation administration may be continuous (for example by way of infusion). A preferred daily oral dose is 20-500 mg and a preferred parenteral dose is in the range 0.1-50 mg.

General Procedures

TLC was performed on silica gel. Chiral HPLC analysis was performed using a 46 mm×250 mm Chiralcel OD column with a 5 cm guard column. The column temperature was maintained at 35° C. A flow rate of 1.0 mL/min was used. A Gilson 115 UV detector at 228 nm was used. The mobile phase consisted of hexanes, ethanol and trifluroacetic acid and the appropriate ratios are listed for each compound. Typically, the product was dissolved in a minimal amount of ethanol and this was diluted with the mobile phase.

In Preparations A to I below, LC-MS/MS was performed using a HP-1100 instrument equipped with a CTC-PAL injector and a 5 Tm, 4×100 mm ThermoQuest, Hypersil BDS-C18 column. An API-3000 (Sciex) MS detector was used. The flow rate was 1.2 mL/min and the mobile phase (gradient) consisted of 10-90% acetonitrile with 90-10% of 4 mM aq. ammonium acetate, both containing 0.2% formic acid. Otherwise, low resolution mass spectra (LRMS) were recorded using a Micromass ZQ spectrometer in ESI posneg switching ion mode (mass range m/z 100-800); and high resolution mass spectra (HRMS) were recorded using a Micromass LCT spectrometer in ES negative ionization mode (mass range m/z 100-1000) with Leucine Enkephalin (C₂₈H₃₇N₅O₇) as internal mass standard.

¹H NMR spectra were recorded using tetramethylsilane as the internal standard.

Processes for the synthesis of compounds of formula (I) are contained in International Patent Application No. PCT/SE01/02657 (WO 02/44145, earliest priority date 1 Dec. 2000, filed 30 Nov. 2001, published 6 Jun. 2002)), relevant information from which is incorporated herein.

Preparation A: Preparation of Compound A

### (i) 3-Chloro-5-methoxybenzaldehyde

3,5-Dichloroanisole (74.0 g, 419 mmol) in THF (200 mL) was added dropwise to magnesium metal (14.2 g, 585 mmol, pre-washed with 0.5 N HCl) in THF (100 mL) at 25° C. After the addition, 1,2-dibromoethane (3.9 g, 20.8 mmol) was added dropwise. The resultant dark brown mixture was heated at reflux for 3 h. The mixture was cooled to 0° C., and N,N-dimethylformamide (60 mL) was added in one portion. The mixture was partitioned with diethyl ether (3×400 mL) and 6N HCl (500 mL). The combined organic extracts were washed with brine (300 mL), dried (Na₂SO₄), filtered and concentrated in vacuo to give an oil. Flash chromatography (2×) on silica gel eluting with Hex:EtOAc (4:1) afforded the sub-title compound (38.9 g, 54%) as a yellow oil.

¹H NMR (300 MHz, CDCl₃) δ 9.90 (s, 1H), 7.53 (s, 1H), 7.38 (s, 1H), 7.15 (s, 1H), 3.87 (s, 3H).

### (ii) 3-Chloro-5-hydroxybenzaldehyde

A solution of 3-chloro-5-methoxybenzaldehyde (22.8 g, 134 mmol; see step (i) above) in CH₂Cl₂ (250 mL) was cooled to 0° C. Boron tribromide (15.8 mL, 167 mmol) was added dropwise over 15 min. After stirring, the reaction mixture for 2 h, H₂O (50 mL) was added slowly. The solution was then extracted with Et₂O (2×100 mL). The organic layers were combined, dried (Na₂SO₄), filtered and concentrated in vacuo. Flash chromatography on silica gel eluting with Hex:EtOAc (4:1) afforded the sub-title compound (5.2 g, 25%).

¹H NMR (300 MHz, CDCl₃) δ 9.85 (s, 1H), 7.35 (s, 1H), 7.20 (s, 1H), 7.10 (s, 1H), 3.68 (s, 1H)

### (iii) 3-Chloro-5-difluoromethoxybenzaldehyde

A solution of 3-chloro-5-hydroxybenzaldehyde (7.5 g, 48 mmol; see step (ii) above) in 2-propanol (250 mL) and 30% KOH (100 mL) was heated to reflux. While stirring, CHClF₂ was bubbled into the reaction mixture for 2 h. The reaction mixture was cooled, acidified with 1N HCl and extracted with EtOAc (2×100 mL). The organics were washed with brine (100 mL), dried (Na₂SO₄), filtered and concentrated in vacuo. Flash chromatography on silica gel eluting with Hex:EtOAc (4:1) afforded the sub-title compound (4.6 g, 46%).

¹H NMR (300 MHz, CDCl₃) δ 9.95 (s, 1H), 7.72 (s, 1H), 7.52 (s, 1H), 7.40 (s, 1H), 6.60 (t, J_{H-F}=71.1 Hz, 1H)

### (iv) Ph(3-Cl)(5-OCHF₂)—(R,S)CH(OTMS)CN

A solution of 3-chloro-5-difluoromethoxybenzaldehyde (4.6 g, 22.3 mmol; see step (iii) above) in CH₂Cl₂ (200 mL) was cooled to 0° C. ZnI₂ (1.8 g, 5.6 mmol) and trimethylsilyl cyanide (2.8 g, 27.9 mmol) were added and the reaction mixture was allowed to warm to room temperature and stirred for 15 h. The mixture was partially concentrated in vacuo yielding the sub-title compound as a liquid, which was used directly in step (v) below without further purification or characterization.

### (v) Ph(3-Cl)(5-OCHF₂)—(R,S)CH(OH)C(NH)OEt

Ph(3-Cl)(5-OCHF₂)—(R,S)CH(OTMS)CN (6.82 g, assume 22.3 mmol; see step (iv) above) was added dropwise to HCl/EtOH (500 mL). The reaction mixture was stirred 15 h, then partially concentrated in vacuo yielding the sub-title compound as a liquid, which was used in step (vi) without further purification or characterization.

### (vi) Ph(3-Cl)(5-OCHF₂)—(R,S)CH(OH)C(O)OEt

Ph(3-Cl)(5-OCHF₂)—(R,S)CH(OH)C(NH)OEt (6.24 g, assume 22.3 mmol; see step (v) above) was dissolved in THF (250 mL), 0.5M H₂SO₄ (400 mL) was added and the reaction was stirred at 40° C. for 65 h, cooled and then partially concentrated in vacuo to remove most of the THF. The reaction mixture was then extracted with Et₂O (3×100 mL), dried (Na₂SO₄), filtered and concentrated in vacuo to afford the sub-title compound as a solid, which was used in step (vii) without further purification or characterization.

### (vii) Ph(3-Cl)(5-OCHF₂)—(R,S)CH(OH)C(O)OH

A solution of Ph(3-Cl)(5-OCHF₂)—(R,S)CH(OH)C(O)OEt (6.25 g, assume 22.3 mmol; see step (vi) above) in 2-propanol (175 mL) and 20% KOH (350 mL) was stirred at room temperature 15 h. The reaction was then partially concentrated in vacuo to remove most of the 2-propanol. The remaining mixture was acidified with 1M H₂SO₄, extracted with Et₂O (3×100 mL), dried (Na₂SO₄) and concentrated in vacuo to give a solid. Flash chromatography on silica gel eluting with CHCl₃:MeOH:concentrated NH₄OH (6:3:1) afforded the ammonium salt of the sub-title compound. The ammonium salt was then dissolved in a mixture of EtOAc (75 mL) and H₂O (75 mL) and acidified with 2N HCl. The organic layer was separated and washed with brine (50 mL), dried (Na₂SO₄) and concentrated in vacuo to afford the sub-title compound (3.2 g, 57% from steps (iv) to (vii)).

¹H NMR (300 MHz, CD₃OD) δ 7.38 (s, 1H), 7.22 (s, 1H), 7.15 (s, 1H), 6.89 (t, J_{H-F}=71.1 Hz, 1H), 5.16 (s, 1H)

### (viii) Ph(3-Cl)(5-OCHF₂)—(R)CH(OH)C(O)OH (a) and Ph(3-Cl)(5-OCHF₂)—(S)CH(OAc)C(O)OH (b)

A mixture of Ph(3-Cl)(5-OCHF₂)—(R,S)CH(OH)C(O)OH (3.2 g, 12.7 mmol; see step (vii) above) and Lipase PS “Amano” (˜2.0 g) in vinyl acetate (125 mL) and MTBE (125 mL) was heated at reflux for 48 h. The reaction mixture was cooled, filtered through Celite® and the filter cake washed with EtOAc. The filtrate was concentrated in vacuo and subjected to flash chromatography on silica gel eluting with CHCl₃:MeOH:concentrated NH₄OH (6:3:1) yielding the ammonium salts of the sub-title compounds (a) and (b). Compound (a) as a salt was dissolved in H₂O, acidified with 2N HCl and extracted with EtOAc. The organic layer was washed with brine, dried (Na₂SO₄), filtered and concentrated in vacuo to afford the sub-title compound (a) (1.2 g, 37%).

For Sub-Title Compound (a)

¹H NMR (300 MHz, CD₃OD) δ 7.38 (s, 1H), 7.22 (s, 1H), 7.15 (s, 1H), 6.89 (t, J_{H-F}=71.1 Hz, 1H), 5.17 (s, 1H)

### (ix) Ph(3-Cl)(5-OCHF₂)—(R)CH(OH)C(O)-Aze-Pab(Teoc)

To a solution of Ph(3-Cl)(5-OCHF₂)—(R)CH(OH)C(O)OH (1.1 g, 4.4 mmol; see step (viii) above) and H-Aze-Pab(Teoc) (see international patent application WO 00/42059, 2.6 g, 5.7 mmol) in DMF (50 mL) at 0° C. was added PyBOP (2.8 g, 5.3 mmol) and collidine (1.3 g, 10.6 mmol). The reaction was stirred at 0° C. for 2 h and then at room temperature for an additional 15 h. The reaction mixture was concentrated in vacuo and flash chromatographed on silica gel (3×), eluting first with CHCl₃:EtOH (9:1), then with EtOAc:EtOH (20:1) and finally eluting with CH₂Cl₂:CH₃OH (95:5) to afford the sub-title compound (1.0 g, 37%) as a white solid.

¹H NMR (300 MHz, CD₃OD, mixture of rotamers) δ 7.79-7.85 (d, J=8.7 Hz, 2H), 7.15-7.48 (m, 5H), 6.89 and 6.91 (t, J_{H-F}=71.1 Hz, 1H), 5.12 and 5.20 (s, 1H), 4.75-4.85 (m, 1H), 3.97-4.55 (m, 6H), 2.10-2.75 (m, 2H), 1.05-1.15 (m, 2H), 0.09 (s, 9H)

MS (m/z) 611 (M+1)⁺

### (x) Ph(3-Cl)(5-OCHF₂)—(R)CH(OH)C(O)-Aze-Pab(OMe, Teoc)

Ph(3-Cl)(5-OCHF₂)—(R)CH(OH)C(O)-Aze-Pab(Teoc) (0.40 g, 0.65 mmol; see step (ix) above), was dissolved in 20 mL of acetonitrile and 0.50 g (6.0 mmol) of O-methyl hydroxylamine hydrochloride was added. The mixture was heated at 70° C. for 2 h. The solvent was evaporated and the residue was partitioned between water and ethyl acetate. The aqueous phase was extracted twice more with ethyl acetate and the combined organic phase was washed with water, brine, dried (Na₂SO₄), filtered and evaporated. Yield: 0.41 g (91%).

¹H-NMR (400 MHz; CDCl₃): δ 7.83 (bt, 1H), 7.57 (bs, 1H), 7.47 (d, 2H), 7.30 (d, 2H), 7.20 (m, 1H), 7.14 (m, 1H), 7.01 (m, 1H), 6.53 (t, 1H), 4.89 (s, 1H), 4.87 (m, 1H), 4.47 (m, 2H), 4.4-4.2 (b, 1H), 4.17-4.1 (m, 3H), 3.95 (s, 3H), 3.67 (m, 1H), 2.68 (m, 1H), 2.42 (m, 1H) 0.97 (m, 2H), 0.01 (s, 9H).

### (xi) Compound A

Ph(3-Cl)(5-OCHF₂)—(R)CH(OH)C(O)-Aze-Pab(OMe, Teoc) (0.40 g, 0.62 mmol; see step (x) above), was dissolved in 5 mL of TFA and allowed to react for 30 min. TFA was evaporated and the residue was partitioned between ethyl acetate and NaHCO₃ (aq.). The aqueous phase was extracted twice more with ethyl acetate and the combined organic phase was washed with water, brine, dried (Na₂SO₄), filtered and evaporated. The product was freeze dried from water/acetonitrile. No purification was necessary. Yield: 0.28 g (85%).

¹H-NMR (600 MHz; CDCl₃): δ 7.89 (bt, 1H), 7.57 (d, 2H), 7.28 (d, 2H), 7.18 (m, 1H), 7.13 (m, 1H), 6.99 (m, 1H), 6.51 (t, 1H), 4.88 (s, 1H), 4.87 (m, 1H), 4.80 (bs, 2H), 4.48 (dd, 1H), 4.43 (dd, 1H), 4.10 (m, 1H), 3.89 (s, 3H), 3.68 (m, 1H), 2.68 (m, 1H), 2.40 (m, 1H).

¹³C-NMR (125 MHz; CDCl₃): (carbonyl and/or amidine carbons, rotamers) δ 172.9, 170.8, 152.7, 152.6

HRMS calculated for C₂₂H₂₃ClF₂N₄O₅ (M−H)⁻ 495.1242, found 495.1247

Preparation B: Preparation of Compound B

### (i) 2,6-Difluoro-4[(methylsulfinyl)(methylthio)methyl]benzonitrile

(Methylsulfinyl)(methylthio)methane (7.26 g, 0.0584 mol) was dissolved in 100 mL of dry THF under argon and was cooled to −78° C. Butyllithium in hexane (16 mL 1.6M, 0.0256 mol) was added dropwise with stirring. The mixture was stirred for 15 min. Meanwhile, a solution of 3,4,5-trifluorobenzonitrile (4.0 g, 0.025 mmol) in 100 mL of dry THF was cooled to −78° C. under argon and the former solution was added through a cannula to the latter solution over a period of 35 min. After 30 min, the cooling bath was removed and when the reaction had reached room temperature it was poured into 400 mL of water. The THF was evaporated and the remaining aqueous layer was extracted three times with diethyl ether. The combined ether phase was washed with water, dried (Na₂SO₄) and evaporated. Yield: 2.0 g(30%).

¹H NMR (500 MHz, CDCl₃) δ 7.4-7.25 (m, 2H), 5.01 (s, 1H, diasteromer), 4.91 (s, 1H, diasteromer), 2.88 (s, 3H, diasteromer), 2.52 (s, 3H, diasteromer), 2.49 (s, 3H, diasteromer), 2.34 (s, 3H, diasteromer), 1.72 (broad, 1H)

### (ii) 2,6-Difluoro-4-formylbenzonitrile

2,6-Difluoro-4[(methylsulfinyl)(methylthio)methyl]benzonitrile (2.17 g, 8.32 mmol; see step (i) above) was dissolved in 90 mL of THF and 3.5 mL of concentrated sulfuric acid was added. The mixture was left at room temperature for 3 days and subsequently poured into 450 mL of water. Extraction three times with EtOAc followed and the combined ethereal phase was washed twice with aqueous sodium bicarbonate and with brine, dried (Na₂SO₄) and evaporated. Yield: 1.36 g (98%). The position of the formyl group was established by ¹³C NMR. The signal from the fluorinated carbons at 162.7 ppm exhibited the expected coupling pattern with two coupling constants in the order of 260 Hz and 6.3 Hz respectively corresponding to an ipso and a meta coupling from the fluorine atoms.

¹H NMR (400 MHz, CDCl₃) δ 10.35 (s, 1H), 7.33 (m, 2H)

### (iii) 2,6-Difluoro-4-hydroxymethylbenzonitrile

2,6-Difluoro-4-formylbenzonitrile (1.36 g, 8.13 mmol; see step (ii) above) was dissolved in 25 mL of methanol and cooled on an ice bath. Sodium borohydride (0.307 g, 8.12 mmol) was added in portions with stirring and the reaction was left for 65 min. The solvent was evaporated and the residue was partitioned between diethyl ether and aqueous sodium bicarbonate. The ethereal layer was washed with more aqueous sodium bicarbonate and brine, dried (Na₂SO₄) and evaporated. The crude product crystallised soon and could be used without further purification. Yield: 1.24 g (90%).

¹H NMR (400 MHz, CDCl₃) δ 7.24 (m, 2H), 4.81 (s, 2H), 2.10 (broad, 1H)

### (iv) 4-Cyano-2,6-difluorobenzyl methanesulfonate

To an ice cooled solution of 2,6-difluoro-4-hydroxymethylbenzonitrile (1.24 g, 7.32 mmol; see step (iii) above) and methanesulfonyl chloride (0.93 g, 8.1 mmol) in 60 mL of methylene chloride was added triethylamine (0.81 g, 8.1 mmol) with stirring. After 3 h at 0° C., the mixture was washed twice with 1M HCl and once with water, dried (Na₂SO₄) and evaporated. The product could be used without further purification. Yield: 1.61 g (89%).

¹H NMR (300 MHz, CDCl₃) δ 7.29 (m, 2H), 5.33 (s, 2H), 3.07 (s, 3H)

### (v) 4-Azidomethyl-2,6-difluorobenzonitrile

A mixture of 4-cyano-2,6-difluorobenzyl methanesulfonate (1.61 g, 6.51 mmol; see step (iv) above) and sodium azide (0.72 g, 0.0111 mol) in 10 mL of water and 20 mL of DMF was stirred at room temperature overnight. The resultant was subsequently poured into 200 mL of water and extracted three times with diethyl ether. The combined ethereal phase was washed five times with water, dried (Na₂SO₄) and evaporated. A small sample was evaporated for NMR purposes and the product crystallised. The rest was evaporated cautiously but not until complete dryness. Yield (theoretically 1.26 g) was assumed to be almost quantitative based on NMR and analytical HPLC.

¹H NMR (400 MHz, CDCl₃) δ 7.29 (m, 2H), 4.46 (s, 2H)

### (vi) 4-Aminomethyl-2,6-difluorobenzonitrile

This reaction was carried out according to the procedure described in J. Chem. Res. (M) (1992) 3128. To a suspension of 520 mg of 10% Pd/C (50% moisture) in 20 mL of water was added a solution of sodium borohydride (0.834 g, 0.0221 mol) in 20 mL of water. Some gas evolution resulted. 4-Azidomethyl-2,6-difluorobenzonitrile (1.26 g, 6.49 mmol; see step (v) above) was dissolved in 50 mL of THF and added to the aqueous mixture on an ice bath over 15 min. The mixture was stirred for 4 h, whereafter 20 mL of 2M HCl was added and the mixture was filtered through Celite. The Celite was rinsed with more water and the combined aqueous phase was washed with EtOAc and subsequently made alkaline with 2M NaOH. Extraction three times with methylene chloride followed and the combined organic phase was washed with water, dried (Na₂SO₄) and evaporated. Yield: 0.87 g (80%).

¹H NMR (400 MHz, CDCl₃) δ 7.20 (m, 2H), 3.96 (s, 2H), 1.51 (broad, 2H)

### (vii) 2,6-Difluoro-4-tert-butoxycarbonylaminomethylbenzonitrile

A solution of 4-aminomethyl-2,6-difluorobenzonitrile (0.876 g, 5.21 mmol; see step (vi) above) was dissolved in 50 mL of THF and di-tert-butyl dicarbonate (1.14 g, 5.22 mmol) in 10 mL of THF was added. The mixture was stirred for 3.5 h. The THF was evaporated and the residue was partitioned between water and EtOAc. The organic layer was washed three times with 0.5 M HCl and water, dried (Na₂SO₄) and evaporated. The product could be used without further purification. Yield: 1.38 g (99%).

¹H NMR (300 MHz, CDCl₃) δ 7.21 (m, 2H), 4.95 (broad, 1H), 4.43 (broad, 2H), 1.52 (s, 9H)

### (viii) Boc-Pab(2,6-diF)(OH)

A mixture of 2,6-difluoro-4-tert-butoxycarbonylaminomethylbenzonitrile (1.38 g, 5.16 mmol; see step (vii) above), hydroxylamine hydrochloride (1.08 g, 0.0155 mol) and triethylamine (1.57 g, 0.0155 mol) in 20 mL of ethanol was stirred at room temperature for 36 h. The solvent was evaporated and the residue was partitioned between water and methylene chloride. The organic layer was washed with water, dried (Na₂SO₄) and evaporated. The product could be used without further purification. Yield: 1.43 g (92%).

¹H NMR (500 MHz, CD₃OD) δ 7.14 (m, 2H), 4.97 (broad, 1H), 4.84 (broad, 2H), 4.40 (broad, 2H), 1.43 (s, 9H)

### (ix) Boc-Pab(2,6-diF)xHOAc

This reaction was carried out according to the procedure described by Judkins et al, Synth. Comm. (1998) 4351. Boc-Pab(2,6-diF)(OH) (1.32 g, 4.37 mmol; see step (viii) above), acetic anhydride (0.477 g, 4.68 mmol) and 442 mg of 10% Pd/C (50% moisture) in 100 mL of acetic acid was hydrogenated at 5 atm pressure for 3.5 h. The mixture was filtered through Celite, rinsed with ethanol and evaporated. The residue was freeze-dried from acetonitrile and water and a few drops of ethanol. The sub-title product could be used without further purification. Yield: 1.49 g (99%).

¹H NMR (400 MHz, CD₃OD) δ 7.45 (m, 2H), 4.34 (s, 2H), 1.90 (s, 3H), 1.40 (s, 9H)

### (x) Boc-Pab(2,6-diF)(Teoc)

To a solution of Boc-Pab(2,6-diF)xHOAc (1.56 g, 5.49 mmol; see step (ix) above) in 100 mL of THF and 1 mL of water was added 2-(trimethylsilyl)ethyl p-nitrophenyl carbonate (1.67 g, 5.89 mmol). A solution of potassium carbonate (1.57 g, 0.0114 mol) in 20 mL of water was added dropwise over 5 min. The mixture was stirred overnight. The THF was evaporated and the residue was partitioned between water and methylene chloride. The aqueous layer was extracted with methylene chloride and the combined organic phase was washed twice with aqueous sodium bicarbonate, dried (Na₂SO₄) and evaporated. Flash chromatography on silica gel with heptane/EtOAc=2/1 gave 1.71 g (73%) of pure compound.

¹H NMR (400 MHz, CDCl₃) δ 7.43 (m, 2H), 4.97 (broad, 1H), 4.41 (broad, 2H), 4.24 (m, 2H), 1.41 (s, 9H), 1.11 (m, 2H), 0.06 (s, 9H)

### (xi) Boc-Aze-Pab(2,6-diF)(Teoc)

Boc-Pab(2,6-diF)(Teoc) (1.009 g, 2.35 mmol; see step (x) above) was dissolved in 50 mL of EtOAc saturated with HCl(g). The mixture was left for 10 min., evaporated and dissolved in 18 mL of DMF, and then cooled on an ice bath. Boc-Aze-OH (0.450 g, 2.24 mmol), PyBOP (1.24 g, 2.35 mmol) and lastly diisopropylethyl amine (1.158 g, 8.96 mmol) were added. The reaction mixture was stirred for 2 h and then poured into 350 mL of water and extracted three times with EtOAc. The combined organic phase was washed with brine, dried (Na₂SO₄) and evaporated. Flash chromatography on silica gel with heptane:EtOAc (1:3) gave 1.097 g (96%) of the desired compound.

¹H NMR (500 MHz, CDCl₃) δ 7.46 (m, 2H), 4.65-4.5 (m, 3H), 4.23 (m, 2H), 3.87 (m, 1H), 3.74 (m, 1H), 2.45-2.3 (m, 2H), 1.40 (s, 9H), 1.10 (m, 2H), 0.05 (s, 9H)

### (xii) Ph(3-Cl)(5-OCHF₂)—(R)CH(OH)C(O)-Aze-Pab(2,6-diF)(Teoc)

Boc-Aze-Pab(2,6-diF)(Teoc) (0.256 g, 0.500 mmol; see step (xi) above) was dissolved in 20 mL of EtOAc saturated with HCl(g). The mixture was left for 10 min. and evaporated and dissolved in 5 mL of DMF. Ph(3-Cl)(5-OCHF₂)—(R)CH(OH)C(O)OH (0.120 g, 0.475 mmol; see Preparation A(viii) above), PyBOP (0.263 g, 0.498 mmol) and lastly diisopropylethyl amine (0.245 g, 1.89 mmol) were added. The reaction mixture was stirred for 2 h and then poured into 350 mL of water and extracted three times with EtOAc. The combined organic phase was washed with brine, dried (Na₂SO₄) and evaporated. Flash chromatography on silica gel with EtOAc gave 0.184 g (60%) of the desired sub-title compound.

¹H NMR (400 MHz, CD₃OD, mixture of rotamers) δ 7.55-7.45 (m, 2H), 7.32 (m, 1H, major rotamer), 7.27 (m, 1H, minor rotamer), 7.2-7.1 (m, 2H), 6.90 (t, 1H, major rotamer), 6.86 (t, 1H, minor rotamer), 5.15 (s, 1H, major rotamer), 5.12 (m, 1H, minor rotamer), 5.06 (s, 1H, minor rotamer), 4.72 (m, 1H, major rotamer), 4.6-4.45 (m, 2H), 4.30 (m, 1H, major rotamer), 4.24 (m, 2H), 4.13 (m, 1H, major rotamer), 4.04 (m, 1H, minor rotamer), 3.95 (m, 1H, minor rotamer), 2.62 (m, 1H, minor rotamer), 2.48 (m, 1H, major rotamer), 2.22 (m, 1H, major rotamer), 2.10 (m, 1H, minor rotamer), 1.07 (m, 2H), 0.07 (m, 9H)

### (xiii) Ph(3-Cl)(5-OCHF₂)—(R)CH(OH)C(O)-Aze-Pab(2,6-diF)(OMe,Teoc)

A mixture of Ph(3-Cl)(5-OCHF₂)—(R)CH(OH)C(O)-Aze-Pab(2,6-diF)(Teoc) (64 mg, 0.099 mmol; see step (xii) above) and O-methyl hydroxylamine hydrochloride (50 mg, 0.60 mmol) in 4 mL of acetonitrile was heated at 70° C. for 3 h. The solvent was evaporated and the residue was partitioned between water and EtOAc. The aqueous layer was extracted twice with EtOAc and the combined organic phase was washed with water, dried (Na₂SO₄) and evaporated. The product could be used without further purification. Yield: 58 mg (87%).

¹H NMR (400 MHz, CDCl₃) δ 7.90 (bt, 1H), 7.46 (m, 1H), 7.25-6.95 (m, 5H), 6.51, t, 1H), 4.88 (s, 1H), 4.83 (m, 1H), 4.6-4.5 (m, 2H), 4.4-3.9 (m, 4H), 3.95 (s, 3H), 3.63 (m, 1H), 2.67 (m, 1H), 2.38 (m, 1H), 1.87 (broad, 1H), 0.98 (m, 2H), 0.01, s, 9H)

### (xiv) Compound B

Ph(3-Cl)(5-OCHF₂)—(R)CH(OH)C(O)-Aze-Pab(2,6-diF)(OMe,Teoc) (58 mg, 0.086 mmol; see step (xiii) above) was dissolved in 3 mL of TFA, cooled on an ice bath and allowed to react for 2 h. The TFA was evaporated and the residue dissolved in EtOAc. The organic layer was washed twice with aqueous sodium carbonate and water, dried (Na₂SO₄) and evaporated. The residue was freeze-dried from water and acetonitrile to give 42 mg (92%) of the title compound.

¹H NMR (300 MHz, CDCl₃) δ 7.95 (bt, 1H), 7.2-7.1 (m, 4H), 6.99 (m, 1H), 6.52 (t, 1H), 4.88 (s, 1H), 4.85-4.75 (m, 3H), 4.6-4.45 (m, 2H), 4.29 (broad, 1H), 4.09 (m, 1H), 3.89 (s, 3H), 3.69 (m, 1H), 2.64 (m, 1H), 2.38 (m, 1H), 1.85 (broad, 1H)

¹³C-NMR (100 MHz; CDCl₃): (carbonyl and/or amidine carbons) δ 172.1, 169.8, 151.9

APCI-MS: (M+1)=533/535 m/z

Preparation C: Preparation of Compound C

### (i) (2-Monofluoroethyl) methanesulfonate

To a magnetically stirred solution of 2-fluoroethanol (5.0 g, 78.0 mmol) in CH₂Cl₂ (90 mL) under nitrogen at 0° C. was added triethylamine (23.7 g, 234 mmol) and methanesulfonyl chloride (10.7 g, 93.7 mmol). The mixture was stirred at 0° C. for 1.5 h, diluted with CH₂Cl₂ (100 mL) and washed with 2N HCl (100 mL). The aqueous layer was extracted with CH₂Cl₂ (50 mL) and the combined organic extracts washed with brine (75 mL), dried (Na₂SO₄), filtered and concentrated in vacuo to afford the sub-title compound (9.7 g, 88%) as a yellow oil which was used without further purification.

¹H NMR (300 MHz, CDCl₃) δ 4.76 (t, J=4 Hz, 1H), 4.64 (t, J=4 Hz, 1H), 4.52 (t, J=4 Hz, 1H), 4.43 (t, J=4 Hz, 1H), 3.09 (s, 3H).

### (ii) 3-Chloro-5-monofluoroethoxybenzaldehyde

To a solution of 3-chloro-5-hydroxybenzaldehyde (8.2 g, 52.5 mmol; see Preparation A(ii) above) and potassium carbonate (9.4 g, 68.2 mmol) in DMF (10 mL) under nitrogen was added a solution of (2-monofluoroethyl) methanesulfonate (9.7 g, 68.2 mmol; see step (i) above) in DMF (120 mL) dropwise at room temperature. The mixture was heated to 1001° C. for 5 h and then stirred overnight at room temperature. The reaction was cooled to 0° C., poured into ice-cold 2N HCl and extracted with EtOAc. The combined organic extracts were washed with brine, dried (Na₂SO₄), filtered and concentrated in vacuo. The brown oil was chromatographed on silica gel eluting with Hex:EtOAc (4:1) to afford the sub-title compound (7.6 g, 71%) as a yellow oil.

¹H NMR (300 MHz, CDCl₃) δ 9.92 (s, 1H), 7.48 (s, 1H), 7.32 (s, 1H), 7.21 (s, 1H), 4.87 (t, J=4 Hz, 1H), 4.71 (t, J=3 Hz, 1H), 4.33 (t, J=3 Hz, 1H), 4.24 (t, J=3 Hz, 1H).

### (iii) Ph(3-Cl)(5-OCH₂CH₂F)—(R,S)CH(OTMS)CN

To a solution of 3-chloro-5-monofluoroethoxybenzaldehyde (7.6 g, 37.5 mmol; see step (ii) above) and zinc iodide (3.0 g, 9.38 mmol) in CH₂Cl₂ (310 mL) was added trimethylsilyl cyanide (7.4 g, 75.0 mmol) dropwise at 0° C. under nitrogen. The mixture was stirred at 0° C. for 3 h and at room temperature overnight. The reaction was diluted with H₂O (300 mL), the organic layer was separated, dried (Na₂SO₄), filtered and concentrated in vacuo to afford the sub-title compound (10.6 g, 94%) as a brown oil that was used without further purification or characterisation.

### (iv) Ph(3-Cl)(5-OCH₂CH₂F)—(R,S)CH(OH)C(O)OH

Concentrated hydrochloric acid (100 mL) was added to Ph(3-Cl)(5-OCH₂CH₂F)—(R,S)CH(OTMS)CN (10.6 g, 5.8 mmol; see step (iii) above) and the solution stirred at 100° C. for 3 h. After cooling to room temperature, the reaction was further cooled to 0° C., basified slowly with 3N NaOH (300 mL) and washed with Et₂O (3×200 mL). The aqueous layer was acidified with 2N HCl (80 mL) and extracted with EtOAc (3×300 mL). The combined EtOAc extracts were dried (Na₂SO₄), filtered and concentrated in vacuo to afford the sub-title compound (8.6 g, 98%) as a pale yellow solid that was used without further purification.

R_{f}=0.28 (90:8:2 CHCl₃:MeOH:concentrated NH₄OH)

¹H NMR (300 MHz, CD₃OD) δ 7.09 (s, 1H), 7.02 (s, 1H), 6.93 (s, 1H), 5.11 (s, 1H), 4.77-4.81 (m, 1H), 4.62-4.65 (m, 1H), 4.25-4.28 (m, 1H), 4.15-4.18 (m, 1H).

### (v) Ph(3-Cl)(5-OCH₂CH₂F)—(S)CH(OAc)C(O)OH (a) and Ph(3-Cl)(5-OCH₂CH₂F)—(R)CH(OH)C(O)OH (b)

A solution of Ph(3-Cl)(5-OCH₂CH₂F)—(R,S)CH(OH)C(O)OH (8.6 g, 34.5 mmol; see step (iv) above) and Lipase PS “Amano” (4.0 g) in vinyl acetate (250 mL) and MTBE (250 mL) was heated at 70° C. under nitrogen for 3 d. The reaction was cooled to room temperature and the enzyme removed by filtration through Celite®. The filter cake was washed with EtOAc and the filtrate concentrated in vacuo. Chromatography on silica gel eluting with CHCl₃:MeOH:Et₃N (90:8:2) afforded the triethylamine salt of sub-title compound (a) as a yellow oil. In addition, the triethylamine salt of sub-title compound (b) (4.0 g) was obtained. The salt of sub-title compound (b) was dissolved in H₂O (250 mL), acidified with 2N HCl and extracted with EtOAc (3×200 mL). The combined organic extracts were dried (Na₂SO₄), filtered and concentrated in vacuo to yield the sub-title compound (b) (2.8 g, 32%) as a yellow oil.

Data for Sub-Title Compound (b):

R_{f}=0.28 (90:8:2 CHCl₃:MeOH:concentrated NH₄OH)

¹H NMR (300 MHz, CD₃OD) δ 7.09 (s, 1H), 7.02 (s, 1H), 6.93 (s, 1H), 5.11 (s, 1H), 4.77-4.81 (m, 1H), 4.62-4.65 (m, 1H), 4.25-4.28 (m, 1H), 4.15-4.18 (m, 1H).

### (vi) Compound C

To a solution of Ph(3-Cl)(5-OCH₂CH₂F)—(R)CH(OH)C(O)OH (818 mg, 3.29 mmol; see step (v) above) in DMF (30 mL) under nitrogen at 0° C. was added HAze-Pab(OMe).2HCl (1.43 g, 4.27 mmol, see international patent application WO 00/42059), PyBOP (1.89 g, 3.68 mmol), and DIPEA (1.06 g, 8.23 mmol). The reaction was stirred at 0° C. for 2 h and then at room temperature overnight. The mixture was concentrated in vacuo and the residue chromatographed two times on silica gel, eluting first with CHCl₃:EtOH (15:1) and second with EtOAc:EtOH (20:1) to afford the title compound (880 mg, 54%).

R_{f}=0.60 (10:1 CHCl₃:EtOH)

¹H NMR (300 MHz, CD₃OD, complex mixture of rotamers) δ 7.58-7.60 (d, J=8 Hz, 2H), 7.34 (d, J=7 Hz, 2H), 7.05-7.08 (m, 2H), 6.95-6.99 (m, 1H), 5.08-5.13 (m, 1H), 4.77-4.82 (m, 1H), 4.60-4.68 (m, 1H), 3.99-4.51 (m, 7H), 3.82 (s, 3H), 2.10-2.75 (m, 2H).

¹³C-NMR (150 MHz; CD₃OD): (carbonyl and/or amidine carbons) δ 173.3, 170.8, 152.5.

APCI-MS: (M+1)=493 m/z.

### Preparation of Compound D (Ph(3-Cl)(5-OCHF₂)—(R)CH(OH)C(O)-Aze-Pab) Compound D

Ph(3-Cl)(5-OCHF₂)—(R)CH(OH)C(O)-Aze-Pab(Teoc) (0.045 g, 0.074 mmol; see Preparation A (ix) above), was dissolved in 3 mL of TFA and allowed to react for 1 h. TFA was evaporated and the residue was freeze dried from water/acetonitrile to yield 0.043 g (100%) of the sub-title compound as its TFA salt.

¹H-NMR (400 MHz; CD₃OD) rotamers: δ 7.8-7.75 (m, 2H), 7.55-7.5 (m, 2H), 7.35 (m, 1H, major rotamer), 7.31 (m, 1H, minor rotamer), 7.19 (m, 1H, major rotamer), 7.15 (m, 1H), 7.12 (m, 1H, minor rotamer), 6.89 (t, 1H, major rotamer), 6.87 (t, 1H, minor rotamer), 5.22 (m, 1H, minor rotamer), 5.20 (s, 1H, major rotamer), 5.13 (s, 1H, minor rotamer), 4.80 (m, 1H, major rotamer), 4.6-4.4 (m, 2H), 4.37 (m, 1H, major rotamer), 4.19 (m, 1H, major rotamer), 4.07 (m, 1H, minor rotamer), 3.98 (m, 1H, minor rotamer), 2.70 (m, 1H, minor rotamer), 2.55 (m, 1H, major rotamer), 2.29 (m, 1H, major rotamer), 2.15 (m, 1H, minor rotamer)

¹³C-NMR (100 MHz; CD₃OD): (carbonyl and/or amidine carbons, rotamers) δ 172.6, 172.5, 172.0, 171.7, 167.0

MS (m/z) 465 (M−1)⁻, 467 (M+1)⁺

### Preparation of Compound E (Ph(3-Cl)(5-OCHF₂)—(R)CH(OH)C(O)-Aze-Pab(2,6-diF)) Compound E

Ph(3-Cl)(5-OCHF₂)—(R)CH(OH)C(O)-Aze-Pab(2,6-diF)(Teoc) (81 mg, 0.127 mmol; see Preparation B (xii) above) was dissolved in 0.5 mL of methylene chloride and cooled on an ice bath. TFA (3 mL) was added and the reaction was left for 75 min. The TFA was evaporated and the residue was freeze dried from water and acetonitrile. The crude product was purified by preparative RPLC with CH₃CN:0.1M NH₄OAc (35:65) to produce 39 mg (55%) of the title compound as its HOAc salt, purity: 99%.

¹H NMR (400 MHz, CD₃OD mixture of rotamers) δ 7.5-7.4 (m, 2H), 7.32 (m, 1H, major rotamer), 7.28 (m, 1H, minor rotamer), 7.2-7.1 (m, 3H) 6.90 (t, 1H, major rotamer), 6.86 (t, minor rotamer), 5.15 (s, 1H, major rotamer), 5.14 (m, 1H, minor rotamer), 5.07 (s, 1H, minor rotamer), 4.72 (m, 1H, major rotamer), 4.65-4.45 (m, 2H), 4.30 (m, 1H, major rotamer), 4.16 (m, 1H, major rotamer), 4.03 (m, 1H, minor rotamer), 3.95 (m, 1H, minor rotamer), 2.63 (m, 1H, minor rotamer), 2.48 (m, 1H, major rotamer), 2.21 (m, 1H, major rotamer), 2.07 (m, 1H, minor rotamer), 1.89 (s, 3H)

¹³C-NMR (75 MHz; CD₃OD): (carbonyl and/or amidine carbons, mixture of rotamers) δ 171.9, 171.2, 165.0, 162.8, 160.4

APCI-MS: (M+1)=503/505 m/z.

### Preparation of Compound F (Ph(3-Cl)(5-OCH₂CH₂F)—(R)CH(OH)C(O)-Aze-PabxTFA)

### (i) Ph(3-Cl)(5-OCH₂CH₂F)—(R)CH(OH)C(O)-Aze-Pab(Teoc)

To a solution of Ph(3-Cl)(5-OCH₂CH₂F)—(R)CH(OH)C(O)OH (940 mg, 3.78 mmol; see Preparation C (v) above) in DMF (30 mL) under nitrogen at 0° C. was added HAze-Pab(Teoc)-HCl (2.21 g, 4.91 mmol), PyBOP (2.16 g, 4.15 mmol), and DIPEA (1.22 g, 9.45 mmol). The reaction was stirred at 0° C. for 2 h and then at room temperature for 4 h. The mixture was concentrated in vacuo and the residue chromatographed twice on silica gel, eluting first with CHCl₃:EtOH (15:1) and second with EtOAc:EtOH (20:1) to afford the sub-title compound (450 mg, 20%) as a crushable white foam.

Mp: 80-88° C.

R_{f}=0.60 (10:1 CHCl₃:EtOH)

¹H NMR (300 MHz, CD₃OD, complex mixture of rotamers) δ 7.79 (d, J=8 Hz, 2H), 7.42 (d, J=8 Hz, 2H), 7.05-7.08 (m, 1H), 6.93-6.99 (m, 2H), 5.08-5.13 (m, 1H), 4.75-4.80 (m, 2H), 4.60-4.68 (m, 1H), 3.95-4.55 (m, 8H), 2.10-2.75 (m, 2H), 1.05-1.11 (m, 2H), 0.08 (s, 9H).

APCI-MS: (M+1)=607 m/z.

### (ii) Compound F

Ph(3-Cl)(5-OCH₂CH₂F)—(R)CH(OH)C(O)-Aze-Pab(Teoc) (0.357 g, 0.589 mmol; see step (i) above), was dissolved in 10 mL of TFA and allowed to react for 40 min. TFA was evaporated and the residue was freeze dried from water/acetonitrile to yield 0.33 g (93%) of the title compound as its TFA salt.

¹H-NMR (600 MHz; CD₃OD) rotamers: δ 7.8-7.7 (m, 2H), 7.54 (d, 2H), 7.08 (s, 1H, major rotamer), 7.04 (s, 1H, minor rotamer), 6.99 (s, 1H, major rotamer), 6.95 (s, 1H), 6.92 (s, 1H, minor rotamer), 5.18 (m, 1H, minor rotamer), 5.14 (s, 1H, major rotamer), 5.08 (s, 1H, minor rotamer), 4.80 (m, 1H, major rotamer), 4.73 (m, 1H), 4.65 (m, 1H), 4.6-4.4 (m, 2H), 4.35 (m, 1H, major rotamer), 4.21 (doublet of multiplets, 2H), 4.12 (m, 1H, major rotamer), 4.06 (m, 1H, minor rotamer), 3.99 (m, 1H, minor rotamer), 2.69 (m, 1H, minor rotamer), 2.53 (m, 1H, major rotamer), 2.29 (m, 1H, major rotamer), 2.14 (m, 1H, minor rotamer).

¹³C-NMR (150 MHz; CD₃OD): (carbonyl and/or amidine carbons) δ 172.8, 172.1, 167.4.

ESI-MS+: (M+1)=463 (m/z)

### Preparation of Compound G (Ph(3-Cl)(5-OCHF₂)—(R)CH(OH)C(O)-Aze-Pab(OH))

### (i) Ph(3-Cl)(5-OCHF₂)—(R)CH(OH)C(O)-Aze-Pab(OH, Teoc)

Ph(3-Cl)(5-OCHF₂)—(R)CH(OH)C(O)-Aze-Pab(Teoc) (0.148 g, 0.24 mmol; see Preparation A step (ix) above), was dissolved in 9 mL of acetonitrile and 0.101 g (1.45 mmol) of hydroxylamine hydrochloride was added. The mixture was heated at 70° C. for 2.5 h, filtered through Celite® and evaporated. The crude product (0.145 g; 75% pure) was used directly in the next step without further purification.

### (ii) Ph(3-Cl)(5-OCHF₂)—(R)CH(OH)C(O)-Aze-Pab(OH)

Ph(3-Cl)(5-OCHF₂)—(R)CH(OH)C(O)-Aze-Pab(OH, Teoc) (0.145 g, 0.23 mmol; see step (i) above), was dissolved in 0.5 mL of CH₂Cl₂ and 9 mL of TFA. The reaction was allowed to proceed for 60 minutes. TFA was evaporated and the residue was purified using preparative HPLC. The fractions of interest were pooled and freeze-dried (2×), yielding 72 mg (yield over two steps 62%) of the title compound.

MS (m/z) 482 (M−1)⁻; 484 (M+1)⁺

¹H-NMR (400 MHz; CD₃OD): δ 7.58 (d, 2H), 7.33 (m, 3H), 7.15 (m, 2H), 6.89 (t, 1H major rotamer), 6.86 (t, 1H minor rotamer), 5.18 (s, 1H major rotamer; and m, 1H minor rotamer), 5.12 (s, 1H minor rotamer), 4.77 (m, 1H major rotamer), 4.42 (m, 2H), 4.34 (m, 1H major rotamer), 4.14 (m, 1H major rotamer), 4.06 (m, 1H minor rotamer), 3.95 (m, 1H minor rotamer), 2.66 (m, 1H minor rotamer), 2.50 (m, 1H major rotamer), 2.27 (m, 1H major rotamer), 2.14 (m, 1H minor rotamer)

¹³C-NMR (100 MHz; CD₃OD): (carbonyl and/or amidine carbons, rotamers) δ 172.4, 172.3, 172.0, 171.4 152.3, 152.1

### Preparation of Compound H: Ph(3-Cl)(5-OCHF₂)—(R)CH(OH)C(O)—(S)Aze-Pab(2,6-duF)(OH)

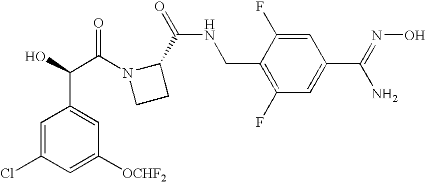

### (i) Boc-(S)Aze-NHCH₂-Ph(2,6-diF, 4-CN)

Boc-(S)Aze-OH (1.14 g, 5.6 mmol) was dissolved in 45 mL of DMF. 4-Aminomethyl-2,6-difluorobenzonitrile (1.00 g, 5.95 mol, see Example 1(xiv) above), PyBOP (3.10 g, 5.95 mmol) and DIPEA (3.95 mL, 22.7 mmol) were added and the solution was stirred at room temperature for 2 h. The solvent was evaporated and the residue was partitioned between H₂O and EtOAc (75 mL each). The aqueous phase was extracted with 2×50 mL EtOAc and the combined organic phase was washed with brine and dried over Na₂SO₄. Flash chromatography (SiO₂, EtOAc/heptane (3/1)) yielded the sub-title compound (1.52 g, 77%) as an oil which crystallized in the refrigerator.

¹H-NMR (400 MHz; CD₃OD): δ 7.19 (m, 2H), 4.65-4.5 (m, 3H), 3.86 (m, 1H), 3.73 (m, 1H), 2.45-2.3 (m, 2H), 1.39 (s, 9H)

### (ii) H—(S)Aze-NHCH₂-Ph(2,6-diF, 4-CN)xHCl

Boc-(S)Aze-NHCH₂-Ph(2,6-diF, 4-CN) (0.707 g, 2.01 mmol, see step (i) above) was dissolved in 60 mL of EtOAc saturated with HCl(g). After stirring at room temperature for 15 minutes, the solvent was evaporated. The residue was dissolved in CH₃CN/H₂O (1/1) and was freeze-dried to give the sub-title compound (0.567 g, 98%) as an off-white amorphous powder.

¹H-NMR (400 MHz; CD₃OD): δ 7.49 (m, 2H), 4.99 (m, 1H), 4.58 (m, 2H), 4.12 (m, 1H), 3.94 (m, 1H), 2.80 (m, 1H), 2.47 (m, 1H)

MS (m/z) 252.0 (M+1)⁺

### (iii) Ph(3-Cl)(5-OCHF₂)—(R)CH(OH)C(O)—(S)Aze-NHCH₂-Ph(2,6-diF, 4-CN)

Ph(3-Cl)(5-OCHF₂)—(R)CH(OH)C(O)OH (0.40 g, 1.42 mmol, see Example l(viii) above) was dissolved in 10 mL of DMF and H—(S)Aze-NHCH₂-Ph(2,6-diF, 4-CN)xHCl (0.43 g, 1.50 mmol, see step (ii) above) and PyBOP (0.779 g, 1.50 mmol) were added, followed by DIPEA (1.0 mL, 5.7 mmol). After stirring at room temperature for 2 h, the solvent was evaporated. The residue was partitioned between H₂O (200 mL) and EtOAc (75 mL). The aqueous phase was extracted with 2×75 mL EtOAc and the combined organic phase was washed with brine and dried over Na₂SO₄. Flash chromatography (SiO₂, EtOAc/heptane (4/1)) yielded the sub-title compound (0.56 g, 81%) as an oil.

¹H-NMR (400 MHz; CD₃OD) rotamers: δ 7.43 (m, 2H), 7.31 (m, 1H, major rotamer), 7.26 (m, 1H, minor rotamer), 7.2-7.1 (m, 2H), 6.90 (t, 1H, major rotamer), 6.86 (t, 1H, minor rotamer), 5.14 (s, 1H, major rotamer), 5.11 (m, 1H, minor rotamer), 5.04 (s, 1H, minor rotamer), 4.71 (m, 1H, major rotamer), 4.6-4.45 (m, 2H), 4.30 (m, 1H, major rotamer), 4.2-3.9 (m, 1H; and 1H, minor rotamer), 2.62 (m, 1H, minor rotamer), 2.48 (m, 1H, major rotamer), 2.21 (m, 1H, major rotamer), 2.09 (m, 1H, minor rotamer)

¹³C-NMR (100 MHz; CD₃OD): (carbonyl carbons) δ 171.9, 171.8

MS (m/z) 484.0, 485.9 (M−1)⁻, 486.0, 487.9 (M+1)⁺

### (iv) Ph(3-Cl)(5-OCHF₂)—(R)CH(OH)C(O)—(S)Aze-Pab(2,6-diF)(OH)

Ph(3-Cl)(5-OCHF₂)—(R)CH(OH)C(O)—(S)Aze-NHCH₂-Ph(2,6-diF, 4-CN) (0.555 g, 1.14 mmol, from step (iii) above) was dissolved in 10 mL of EtOH (95%). To this solution was added hydroxylamine hydrochloride (0.238 g, 3.42 mmol) and Et₃N (0.48 mL, 3.44 mmol). After stirring at room temperature for 14 h, the solvent was removed and the residue was dissolved in EtOAc. The organic phase was washed with brine and H₂O and was dried over Na₂SO₄. The crude product was purified by preparative RPLC with CH₃CN:0.1 M NH₄OAc as eluent, yielding the title compound as an amorphous powder (0.429 g, 72%) after freeze-drying.

¹H-NMR (400 MHz; CD₃OD) rotamers: δ 7.35-7.1 (m, 5H), 6.90 (t, 1H, major rotamer), 6.85 (t, 1H, minor rotamer), 5.15 (s, 1H, major rotamer), 5.12 (m, 1H, minor rotamer), 5.08 (s, 1H, minor rotamer), 4.72 (m, 1H, major rotamer), 4.6-4.4 (m, 2H), 4.30 (m, 1H, major rotamer), 4.12 (m, 1H, major rotamer), 4.04 (m, 1H, minor rotamer), 3.94 (m, 1H, minor rotamer), 2.62 (m, 1H, minor rotamer), 2.48 (m, 1H, major rotamer), 2.22 (m, 1H, major rotamer), 2.10 (m, 1H, minor rotamer)

¹³C-NMR (100 MHz; CD₃OD): (carbonyl and amidine carbons, rotamers) δ 172.4, 171.9, 171.0, 152.3, 151.5

MS (m/z) 517.1, 519.0 (M−1)⁻, 519.1, 521.0 (M+1)⁺

### Preparation of Compound J (Ph(3-Cl)(5-OCH₂CHF₂)—(R)CH(OH)C(O)-Aze-Pab(OH))

### (i) Ph(3-Cl)(5-OCH₂CHF₂)—(R)CH(OH)C(O)-Aze-Pab(Z)

Boc-Aze-Pab(Z) (see international patent application WO 97/02284, 92 mg, 0.197 mmol) was dissolved in 10 mL of EtOAc saturated with HCl(g) and allowed to react for 10 min. The solvent was evaporated and the residue was mixed with Ph(3-Cl)(5-OCH₂CHF₂)—(R)CH(OH)C(O)OH (50 mg, 0.188 mmol; see Preparation C (v) above), PyBOP (109 mg, 0.209 mmol) and finally diisopropylethyl amine (96 mg, 0.75 mmol) in 2 mL of DMF. The mixture was stirred for 2 h and then poured into 50 mL of water and extracted three times with EtOAc. The combined organic phase was washed with water, dried (Na₂SO₄) and evaporated. The crude product was flash chromatographed on silica gel with EtOAc:MeOH (9:1). Yield: 100 mg (87%).

¹H NMR (300 MHz, CD₃OD, mixture of rotamers) δ 7.85-7.75 (m, 2H), 7.45-7.25 (m, 7H), 7.11 (m, 1H, major rotamer), 7.08 (m, 1H, minor rotamer), 7.05-6.9 (m, 2H), 6.13 (bt, 1H), 5.25-5.05 (m, 3H), 4.77 (m, 1H, partially hidden by the CD₃OH signal), 4.5-3.9 (m, 7H), 2.64 (m, 1H, minor rotamer), 2.47 (m, 1H, major rotamer), 2.25 (m, 1H, major rotamer), 2.13 (m, 1H, minor rotamer)

### (ii) Ph(3-Cl)(5-OCH₂CHF₂)—(R)CH(OH)C(O)-Aze-Pab(OH)

Hydroxylamine hydrochloride (65 mg, 0.94 mmol) and triethylamine (0.319 g, 3.16 mmol) were mixed in 8 mL of THF and sonicated for 1 h at 40° C. Ph(3-Cl)(5-OCH₂CHF₂)—(R)CH(OH)C(O)-Aze-Pab(Z) (96 mg, 0.156 mmol; see step (i) above) was added with 8 mL more of THF. The mixture was stirred at 40° C. for 4.5 days. The solvent was evaporated and the crude product was purified by preparative RPLC with CH₃CN:0.1M NH₄OAc (40:60).

Yield: 30 mg (38%). Purity: 99%.

¹H NMR (300 MHz, CD₃OD, mixture of rotamers) δ 7.6-7.55 (m, 2H), 7.35-7.3 (m, 2H), 7.12 (m, 1H, major rotamer), 7.09 (m, 1H, minor rotamer), 7.05-6.9 (m, 2H), 6.15 (triplet of multiplets, 1H), 5.15 (m, 1H, minor rotamer), 5.13 (s, 1H, major rotamer), 5.08 (s, 1H, minor rotamer), 4.77 (m, 1H, major rotamer), 4.5-4.2 (m, 5H), 4.08 (m, 1H, major rotamer), 3.97 (m, 1H, minor rotamer), 2.66 (m, 1H, minor rotamer), 2.50 (m, 1H major rotamer), 2.27 (m, 1H, major rotamer), 2.14 (m, 1H, minor rotamer).

¹³C-NMR (100 MHz; CD₃OD): (carbonyl and/or amidine carbons, mixture of rotamers) δ 172.8, 172.2, 171.4, 159.1, 158.9, 154.2.

APCI-MS: (M+1)=497/499 m/z

### Methods 1 and 2: Preparation of Salts of Compound A

### Method 1: General Method for Salt Preparation

The following generic method was employed to prepare salts of Compound A: 200 mg of Compound A (see Preparation A above) was dissolved in 5 mL of MeOH. To this solution was added a solution of the relevant acid (1.0 molar equivalent) dissolved in 5 mL of MeOH. After stirring for 10 minutes at room temperature, the solvent was removed by way of a rotary evaporator. The remaining solid material was re-dissolved in 8 mL of acetonitrile:H₂O (1:1). Freeze-drying afforded colorless amorphous material in each case.

Acids Employed:

(1S)-(+)-10-camphorsulfonic
malic
cyclohexylsulphamic
phosphoric
dimethylphosphoric
p-toluenesulphonic
L-lysine
L-lysine hydrochloride
saccharinic
methanesulphonic
hydrochloric

Appropriate characterising data are shown in Table 1.

TABLE 1

| | | | | δ ppm (MeOD) |
|---|---|---|---|---|
| | | | | H18, H19, H24 |
| | Mw | Mw | | (see structure at end |
| Salt | acid | salt | LRMS | of Method 9 below) |
| (1S)-(+)-10- | 232.20 | 729.20 | 230.9 | 7.57, 7.68, 3.97 |
| camphorsulfonate | | | 495.1 | |
| | | | 497.0 | |
| | | | 727.3 | |
| maleate | 116.07 | 612.97 | 114.8 | 7.45, 7.64, 3.89 |
| | | | 495.1 | |
| | | | 497.0 | |
| cyclohexylsulphamate | 179.24 | 676.14 | 177.9 | 7.44, 7.64, 3.89 |
| | | | 495.1 | |
| | | | 496.9 | |
| | | | 674.3 | |
| | | | 676.1 | |
| phosphate | 97.99 | 594.89 | 495.1 | 7.37, 7.61, 3.84 |
| | | | 497.0 | |
| | | | 593.1 | |
| dimethylphosphate | 126.05 | 622.95 | 124.9 | 7.50, 7.66, 3.92 |
| | | | 495.1 | |
| | | | 497.0 | |
| | | | 621.2 | |
| | | | 623.0 | |
| p-toluenesulphonate | 172.20 | 669.10 | 170.9 | 7.54, 7.71, 3.95 |
| | | | 495.1 | |
| | | | 497.0 | |
| L-lysine | 146.19 | 643.09 | 145.0 | 7.36, 7.60, 3.83 |
| | | | 495.1 | |
| | | | 497.0 | |
| L-lysine hydrochloride | 182.65 | 679.55 | 495.1 | 7.36, 7.60, 3.83 |
| | | | 497.0 | |
| | | | 531.1 | |
| | | | (HCl) | |
| saccharinate | 183.19 | 680.09 | 181.9 | 7.44, 7.64, 3.89 |
| | | | 495.1 | |
| | | | 497.0 | |
| methanesulphonate | 96.11 | 593.01 | 495.1 | 7.57, 7.68, 3.97 |
| | | | 497.0 | |
| | | | 591.2 | |
| | | | 593.1 | |
| hydrochloride | 36.46 | 533.36 | 495.1 | 7.55, 7.67, 3.95 |
| | | | 496.9 | |
| | | | 531.1 | |
| | | | 532.5 | |
| | | | 535.2 | |

All salts formed in this Method were amorphous.

### Method 2

Further amorphous salts of Compound A were made using analogous techniques to those described in Method 1 above from the following acids:
hydrobromic acid (1:1 salt)
hydrochloric acid (1:1 salt)
sulphuric acid (1:0.5 salt)
1,2-ethanedisulfonic acid (1:0.5 salt)
1S-camphorsulfonic acid (1:1 salt)
(+/−)-camphorsulfonic acid (1:1 salt)
ethanesulfonic acid (1:1 salt)
nitric acid (1:1 salt)
toluenesulfonic acid (1:1 salt)
methanesulfonic acid (1:1 salt)
p-xylenesulfonic acid (1:1 salt)
2-mesitylenesulfonic acid (1:1 salt)
1,5-naphthalenesulfonic acid (1:0.5 salt)
naphthalenesulfonic acid (1:1 salt)
benzenesulfonic acid (1:1 salt)
saccharinic acid (1:1 salt)
maleic acid (1:1 salt)
phosphoric acid (1:1 salt)
D-glutamic acid (1:1 salt)
L-glutamic acid (1:1 salt)
D,L-glutamic acid (1:1 salt)
L-arginine (1:1 salt)
L-lysine (1:1 salt)
L-lysine hydrochloride (1:1 salt)
glycine (1:1 salt)
salicylic acid (1:1 salt)
tartaric acid (1:1 salt)
fumaric acid (1:1 salt)
citric acid (1:1 salt)
L-(−)-malic acid (1:1 salt)
D,L-malic acid (1:1 salt)
D-gluconic acid (1:1 salt)

### Method 3: Preparation of Amorphous Compound A, ethanesulfonic acid salt

Compound A (203 mg; see Preparation A above) was dissolved in ethanol (3 mL) and ethanesulfonic acid (1 eq., 95%, 35 μL) was added to the solution. The mixture was stirred for a few minutes, and then the solvent was evaporated. The resulting oil was slurried in iso-octane and evaporated to dryness until a solid material was obtained. Finally, the substance was re-slurried in iso-octane and the solvent evaporated again resulting in a white, dry, amorphous solid. The substance was vacuum dried at 40° C. overnight.

### Methods 4 to 9: Preparation of Crystalline Compound A, ethanesulfonic acid salt

### Method 4: Crystallisation of Amorphous Material

Amorphous Compound A, ethanesulfonic acid salt (17.8 mg; see Method 3 above) was slurried in methyl iso-butyl ketone (600 μL). After 1 week, crystalline needles were observed, which were filtered off and air-dried.

### Methods 5 to 7: Reaction Crystallisations (without Anti-solvent)

### Method 5

Compound A (277 mg; see Preparation A above) was dissolved in methyl iso-butyl ketone (3.1 mL). Ethanesulfonic acid was added (1 eq., 95%, 48 μL). Precipitation of amorphous ethanesulfonate salt occurred immediately. More methyl iso-butyl ketone (6 mL) was added and the slurry was treated with ultrasound. Finally, a third portion of methyl iso-butyl ketone (3.6 mL) was added and then the slurry was left overnight with stirring (magnetic stirrer). The next day, the substance had transformed into crystalline needles. The slurry was filtered off, washed with methyl iso-butyl ketone (0.5 mL) and air dried.

### Method 6

Compound A (236 mg; see Preparation A above) was dissolved at room temperature in methyl iso-butyl ketone (7 mL). Ethanesulfonic acid (1 eq., 41 μL) was mixed with 2 mL of methyl iso-butyl ketone in a vial. The solution of Compound A was seeded with crystalline Compound A, ethanesulfonic acid salt (see Methods 4 and 5 above). Then, 250 μL of the methyl iso-butyl ketone solution of ethanesulfonic acid was added in portions over 45 minutes. The solution was seeded again, and the temperature was increased to 30° C. Then, 500 μL of the methyl iso-butyl ketone solution was added over approximately 1 hour. The resulting slurry was left overnight before a final amount of the methyl iso-butyl ketone/acid solution was added over 20 minutes. The vial was rinsed with 1.5 mL of methyl iso-butyl ketone, which was added to the slurry. After a further 6 hours, the crystals were filtered off, washed with methyl iso-butyl ketone (2 mL) and dried under reduced pressure at 40° C. A total of 258 mg of crystalline salt was obtained which corresponds to a yield of approximately 87%.

### Method 7

Compound A (2.36 g; see Preparation A above) was dissolved in methyl iso-butyl ketone (90 mL). Seed crystals (10 mg) of Compound A, ethanesulfonic acid salt (see Methods 4 to 6 above) were added to the solution, and then ethanesulfonic acid (40 μL) was added in two portions. Further seed crystals (12 mg) and two portions of ethanesulfonic acid (2×20 μL) were then added. The slurry was diluted with methyl iso-butyl ketone (15 mL) before the addition of ethanesulfonic acid was continued. A total amount of 330 μL ethanesulfonic acid was added, in portions, over 1 hour. A small amount of seed crystals was added and, finally, the slurry was left overnight with stirring. The next day, the crystals were filtered off, washed with methyl iso-butyl ketone (2×6 mL) and dried under reduced pressure at 40° C. After drying, a total of 2.57 g of white, crystalline product was obtained corresponding to a yield of 89%.

### Methods 8 and 9: Reaction Crystallizations (with Anti-Solvent)

### Method 8

Compound A (163 mg; see Preparation A above) was dissolved in iso-propanol (1.2 mL). The solution was heated to 35° C. Ethanesulfonic acid was added (28 μL). Then, ethyl acetate (4.8 mL) was added and the solution was seeded with crystalline Compound A, ethanesulphonic acid salt (see Methods 4 to 7 above). Crystallization started almost immediately. The slurry was left for about 80 minutes at 35° C. before being allowed to cool to ambient temperature (21° C.). Two hours later, the crystals were filtered off, washed three times with ethyl acetate (3×0.4 mL), and dried under reduced pressure at 40° C. A total of 170 mg of crystalline title product was obtained which corresponds to a yield of approximately 82%.

### Method 9

Compound A (20.0 g; see Preparation A above) was dissolved in iso-propanol (146.6 mL) at 40° C. and ethanesulfonic acid (3.46 mL, 95%, 1 eq.) was added to the solution. To the resulting clear solution, seed crystals of Compound A, ethanesulfonic acid salt were added (50 mg; see Methods 4 to 8 above). Then, ethyl acetate (234 mL) was added over 10 minutes. The resulting slightly opaque solution was seeded once more (70 mg) and left for one hour at 40° C. with stirring to allow for crystallization to start. After this, a total of 352 mL of ethyl acetate was added at a constant rate over one hour. When all of the ethyl acetate had been added, the slurry was left for 1 hour, before being cooled to 21° C. over 2 hours. The crystallization was allowed to continue for 1 hour at 21° C. before the crystals were filtered off, washed twice with ethyl acetate (50 mL+60 mL) and finally, dried under reduced pressure at 40° C. overnight. A total of 21.6 g of a white, crystalline salt was obtained, corresponding to a yield of approximately 90%.

Compound A, ethanesulfonic acid salt was characterised by NMR as follows: 23 mg of the salt was dissolved in deuterated methanol (0.7 mL) troscopy. A combination of 1D (¹H, ¹³C and selective NOE) and 2D (gCOSY, gHSQC and gHMBC) NMR experiments were used. All data were in good agreement with the theoretical structure of the salt, shown below. The molecule exists in two conformations in methanol. Based on the integral of the peak assigned to H5 (dominant conformer) and peak assigned to H5′ (other conformer), the ratio between the two conformers was found to be 70:30. H22 could not be observed as these protons were in fast exchange with the solvent CD₃OD.

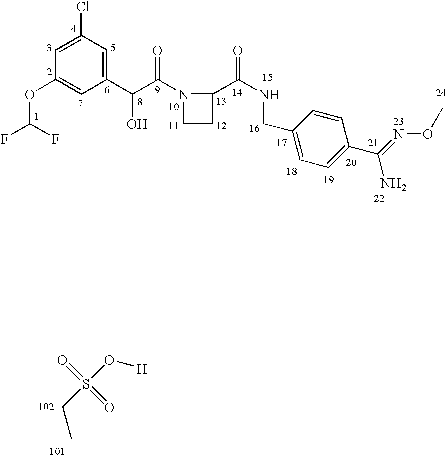

Both the proton and the carbon resonance corresponding to position 1 are split due to the spin-coupling with the two fluorine nuclei in that position. The coupling constants are ²J_{HF}=73 Hz and ¹J_{CF}=263 Hz.

¹H and ¹³C NMR chemical shift assignment and proton-proton correlations are shown in Table 2.

TABLE 2

| Atom | | ¹³C shift/ | ¹H shift/ppm^{b} and | |
|---|---|---|---|---|
| No. | Type | ppm^{a} | multiplicity^{c} | J_{HH}/Hz |
| 1 | CH | 117.5^{e} | 6.90 (t) | 73 (²J_{HF}) |
| 1′ | | 117.5^{e} | 6.88 (t) | |
| 2 | C | 153.5 | | |
| 2′ | | 153.5 | | |
| 3 | CH | 120.0 | 7.15 (s) | |
| 3′ | | 119.7 | 7.13 (s) | |
| 4 | C | 136.2 | | |
| 4′ | | 135.9 | | |
| 5 | CH | 125.0 | 7.36 (s) | |
| 5′ | | 124.9 | 7.31 (s) | |
| 6 | C | 144.5 | | |
| 6′ | | 145.3 | | |
| 7 | CH | 117.3 | 7.20 (s) | |
| 7′ | | 117.2 | 7.15 (s) | |
| 8 | CH | 72.0 | 5.20 (s) | |
| 8′ | | 74.0 | 5.12 (s) | |
| 9 | CO | 173.1 | | |
| 9′ | | 173.8 | | |
| 11 | CH₂ | 51.6 | a: 4.38 (m) | |
| | | | b: 4.21 (m) | |
| 11′ | | 49.0 | a: 4.06 (m) | |
| | | | b: 3.99 (m) | |
| 12 | CH₂ | 21.7 | a: 2.55 (m) | |
| | | | b: 2.29 (m) | |
| 12′ | | 23.2 | a: 2.70 (m) | |
| | | | b: 2.15 (m) | |
| 13 | CH | 63.1 | 4.80 (m) | |
| 13′ | | 66.2 | 5.22 (m) | |
| 14 | CO | 172.9 | | |
| 14′ | | 173.6 | | |
| 15 | NH | | 8.76 (t, br) | 5.2 |
| 15′ | | | 8.79 (t, br) | 5.2 |
| 16 | CH₂ | 43.5 | 4.59 (AB-pattern) | 15.9 |
| | | | 4.46 (AB-pattern) | 15.9 |
| 16′ | | 43.6 | 4.53 (AB-pattern) | 15.9 |
| | | | 4.49 (AB-pattern) | 15.9 |
| 17 | C | 146.9 | | |
| 17′ | | 147.0 | | |
| 18 | CH | 129.1 | 7.56 (d) | 7.8 |
| 18′ | | 129.1 | 7.57 (d) | 7.8 |
| 19 | CH | 129.2 | 7.67 (d) | 7.8 |
| 19′ | | 129.4 | 7.70 (d) | 7.8 |
| 20 | C | 124.9 | — | |
| 20′ | | 124.9 | | |
| 21 | C | 162.4 | | |
| 21′ | | 162.3 | | |
| 22 | NH₂ | | Not observed | |
| 24 | CH₃ | 64.8 | 3.96 (s) | |
| 101 | CH3 | | 1.28 (t) | 7.4 |
| 102 | CH2 | | 2.77 (m) | 7.4 |

^{a}Relative to the solvent resonance at 49.0 ppm.
^{b}Relative to the solvent resonance at 3.30 ppm.
^{c}s = singlet, t = triplet, m = multiplet, br = broad, d = doublet
^{d}Obtained in the gCOSY experiment.
^{e}The resonance is a triplet due to coupling with the two fluorine nuclei.
¹J_{CF} = 263 Hz.

HRMS calculated for C₂₄H₂₉ClF₂N₄O₈S (M−H)hu − 605.1284, found 605.1296.

Crystals of Compound A, ethanesulfonic acid salt (obtained by way of one or more of Examples 4 to 9 above) were analyzed by XPAD and the results are tabulated below (Table 3) and are shown in FIG. 1.

TABLE 3

| d value (Å) | Intensity (%) | Intensity |
|---|---|---|
| 16.5 | 10 | m |
| 12.2 | 74 | vs |
| 11.0 | 4 | w |
| 9.0 | 33 | s |
| 8.3 | 3 | vw |
| 7.6 | 6 | w |
| 6.4 | 4 | w |
| 6.2 | 12 | m |
| 6.0 | 7 | m |
| 5.9 | 10 | m |
| 5.5 | 15 | m |
| 5.4 | 100 | vs |
| 5.1 | 7 | m |
| 4.66 | 29 | s |
| 4.60 | 36 | s |
| 4.31 | 57 | s |
| 4.25 | 18 | m |
| 4.19 | 20 | m |
| 4.13 | 12 | m |
| 4.00 | 12 | m |
| 3.87 | 13 | m |
| 3.83 | 6 | w |
| 3.76 | 7 | m |
| 3.72 | 6 | w |
| 3.57 | 9 | m |
| 3.51 | 7 | m |
| 3.47 | 5 | w |
| 3.39 | 3 | vw |
| 3.31 | 11 | m |
| 3.26 | 10 | m |
| 3.21 | 8 | m |
| 3.16 | 4 | w |
| 3.03 | 8 | m |
| 2.78 | 4 | w |
| 2.74 | 5 | w |
| 2.67 | 3 | vw |
| 2.56 | 5 | w |
| 2.50 | 5 | w |
| 2.46 | 7 | m |
| 2.34 | 4 | w |
| 2.21 | 5 | w |
| 2.00 | 3 | vw |
| 1.98 | 3 | vw |

DSC showed an endotherm with an extrapolated melting onset temperature of ca. 131° C. TGA showed a decrease in mass of ca. 0.2% (w/w) around the melting point. DSC analysis repeated with a sample of lower solvent content showed a melting onset temperature of ca. 144° C.

### Method 10: Preparation of Amorphous Compound A, benzenesulfonic acid salt

Compound A (199 mg; see Preparation A above) was dissolved in ethanol (2 mL). Benzenesulfonic acid (1 eq. 90%, 70 mg) was dissolved in ethanol (1 mL) in a vial. The ethanol solution of the acid was added to the solution of Compound A and the vial was rinsed with 1 mL ethanol, which was then added to the mixture. The mixture was stirred for a few minutes, and then the ethanol was evaporated until an oil was formed. Ethyl acetate (3 mL) was added and the solvent was evaporated again to dryness. An amorphous solid was formed.

### Methods 11 to 13: Preparation of Crystalline Compound A, benzenesulfonic acid salt

### Method 11: Crystallisation of Amorphous Material

Amorphous Compound A benzenesulfonic acid salt (20.7 mg; see Method 10 above) was slurried in ethyl acetate (600 TL). After 5 days, crystalline needles were observed in the slurry.

### Methods 12 and 13: Reaction Crystallisations

### Method 12

Compound A (128 mg; see Preparation A above) was dissolved in ethyl acetate (3 mL). The solution was seeded with the slurry from Method 11 above. Then, benzenesulfonic acid was added (1 eq., 90%, 45 mg). Precipitation of benzenesulphonic acid salt occurred immediately. iso-Propanol was added to the slurry (0.8 mL) and the mixture was seeded again. Two days later, the substance had transformed into crystalline needles. The slurry was filtered off, washed with ethyl acetate (3×0.2 mL) and dried for a short time under vacuum at 40° C. A total of approximately 140 mg of white solid was obtained.

### Method 13

Compound A (246 mg; see Preparation A above) was dissolved in iso-propanol (1.52 mL). Benzenesulfonic acid was added (88 mg, 90%). To the clear solution, ethyl acetate was added (3 mL), and then the mixture was seeded to initiate crystallisation. After 1 hour, more ethyl acetate was added (2.77 mL). Finally, the slurry was allowed to crystallise overnight before the crystals were filtered off, washed with ethyl acetate (3×0.3 mL) and dried at 40° C. under vacuum. A total of 279 mg salt was obtained which corresponds to a yield of approximately 86%.

Compound A, benzenesulfonic acid salt was characterised by NMR as follows: 20 mg of the salt was dissolved in deuterated methanol (0.7 mL). A combination of 1D (¹H, ¹³C and selective NOE) and 2D (gCOSY, gHSQC and gHMBC) NMR experiments were used. All data were in good agreement with the theoretical structure of the salt, shown below. The molecule exists in two conformations in methanol. Based on the integral of the peak assigned to H12 (dominant conformer) and peak assigned to H12′ (other conformer), the ratio between the two conformers was found to be 70:30. H22 could not be observed as these protons were in fast exchange with the solvent CD₃OD.

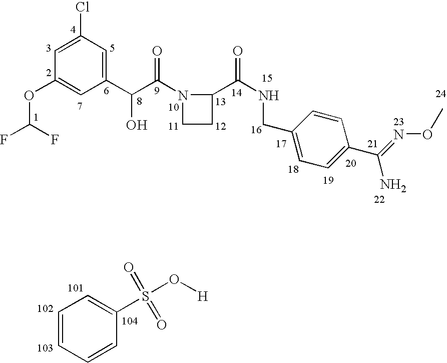

Both the proton and the carbon resonance corresponding to position 1 are split due to the spin-coupling with the two fluorine nuclei in that position. The coupling constants are ²J_{HF}=74 Hz and ¹J_{CF}=260 Hz.

¹H and ¹³C NMR chemical shift assignment and proton-proton correlations are shown in Table 4.

TABLE 4

| Atom | | | ¹H shift/ppm^{b} and | |
|---|---|---|---|---|
| No. | Type | ¹³C shift/ppm^{a} | multiplicity^{c} | J_{HH}/Hz |
| 1 | CH | 117.5^{e} | 6.89 (t) | 74 (²J_{HF}) |
| 1′ | | 117.5^{e} | 6.87 (t) | |
| 2 | C | 153.5 | | |
| 2′ | | 153.5 | | |
| 3 | CH | 120.1 | 7.15 (s) | |
| 3′ | | 119.7 | 7.12 (s) | |
| 4 | C | 136.2 | | |
| 4′ | | 135.9 | | |
| 5 | CH | 125.1 | 7.35 (s) | |
| 5′ | | 124.9 | 7.31 (s) | |
| 6 | C | 144.5 | | |
| 6′ | | 145.3 | | |
| 7 | CH | 117.3 | 7.20 (s) | |
| 7′ | | 117.2 | 7.14 (s) | |
| 8 | CH | 72.8 | 5.20 (s) | |
| 8′ | | 74.0 | 5.12 (s) | |
| 9 | CO | 173.1 | | |
| 9′ | | 173.8 | | |
| 11 | CH₂ | 51.6 | a: 4.37 (m) | |
| | | | b: 4.20 (m) | |
| 11′ | | 49.0 | a: 4.05 (m) | |
| | | | b: 3.98 (m) | |
| 12 | CH₂ | 21.7 | a: 2.53 (m) | |
| | | | b: 2.28 (m) | |
| 12′ | | 23.2 | a: 2.69 (m) | |
| | | | b: 2.14 (m) | |
| 13 | CH | 63.1 | 4.79 (m) | |
| 13′ | | 66.2 | 5.22 (m) | |
| 14 | CO | 172.9 | | |
| 14′ | | 173.6 | | |
| 15 | NH | | 8.75 (t, br) | 5.3 |
| 15′ | | | 8.78 (t, br) | 5.3 |
| 16 | CH₂ | 43.5 | 4.59 (AB-pattern) | 16.0 and 5.2 |
| | | | 4.44 (AB-pattern) | 16.0 and 4.8 |
| 16′ | | 43.6 | 4.51 (AB-pattern) | 16.0 |
| | | | 4.46 (AB-pattern) | 16.0 |
| 17 | C | 146.9 | | |
| 17′ | | 147.0 | | |
| 18 | CH | 129.2 | 7.54 (d) | 8.3 |
| 18′ | | 129.2 | 7.56 (d) | 8.3 |
| 19 | CH | 129.3 | 7.66 (d) | 8.3 |
| 19′ | | 129.4 | 7.69 (d) | 8.3 |
| 20 | C | 124.9 | — | |
| 20′ | | 124.9 | | |
| 21 | C | 162.4 | | |
| 21′ | | 162.4 | | |
| 22 | NH₂ | | Not observed | |
| 24 | CH₃ | 64.8 | 3.95 (s) | |
| 101 | CH | 126.9 | 7.81 (m) | |
| 102 | CH | 129.1 | 7.41 (m) | |
| 103 | CH | 131.2 | 7.42 (m) | |
| 104 | C | 146.4 | | |

^{a}Relative to the solvent resonance at 49.0 ppm.
^{b}Relative to the solvent resonance at 3.30 ppm.
^{c}s = singlet, t = triplet, m = multiplet, br = broad, d = doublet.
^{d}Obtained in the gCOSY experiment.
^{e}The resonance is a triplet due to coupling with the two fluorine nuclei.
¹J_{CF} = 260 Hz.
^{f}connectivity difficult to determine due to overlap between resonance 102 and 103

HRMS calculated for C₂₈H₂₉ClF₂N₄O₈S (M−H)⁻ 653.1284, found 653.1312.

Crystals of Compound A, benzenesulfonic acid salt (obtained by way of one or more of Examples 11 to 13 above) were analyzed by XRPD and the results are tabulated below (Table 5) and are shown in FIG. 2.

TABLE 5

| d value (Å) | Intensity (%) | Intensity |
|---|---|---|
| 14.2 | 12 | m |
| 12.6 | 55 | s |
| 10.2 | 49 | s |
| 7.5 | 8 | m |
| 6.4 | 5 | w |
| 6.3 | 30 | s |
| 6.1 | 5 | w |
| 5.9 | 100 | vs |
| 5.7 | 20 | m |
| 5.4 | 9 | m |
| 5.3 | 11 | m |
| 5.1 | 10 | m |
| 4.96 | 3 | vw |
| 4.83 | 27 | s |
| 4.73 | 72 | vs |
| 4.54 | 23 | s |
| 4.50 | 10 | m |
| 4.35 | 28 | s |
| 4.30 | 38 | s |
| 4.24 | 24 | s |
| 4.17 | 28 | s |
| 4.09 | 60 | vs |
| 4.08 | 61 | vs |
| 3.96 | 29 | s |
| 3.91 | 15 | m |
| 3.77 | 22 | s |
| 3.62 | 11 | m |
| 3.52 | 20 | m |
| 3.31 | 44 | s |
| 3.19 | 8 | m |
| 3.15 | 11 | m |
| 3.09 | 8 | m |
| 3.00 | 7 | m |
| 2.89 | 3 | vw |
| 2.86 | 4 | w |
| 2.79 | 7 | m |
| 2.76 | 6 | w |
| 2.72 | 5 | w |
| 2.59 | 6 | w |
| 2.56 | 9 | m |
| 2.54 | 9 | m |
| 2.49 | 7 | m |
| 2.38 | 8 | m |
| 2.16 | 4 | w |
| 2.03 | 3 | vw |

DSC showed an endotherm with an extrapolated melting onset temperature of ca. 152° C. TGA showed a decrease in mass of ca. 0.1% (w/w) around the melting point.

### Method 14: Preparation of Amorphous Compound A, n-propanesulfonic acid salt

Compound A (186 mg; see Preparation A above) was dissolved in iso-propanol (1.39 mL) and n-propanesulfonic acid (1 eq., 95%, 39 TL) was added. Ethyl acetate (5.6 mL) was added and the solvent was evaporated until a dry, amorphous solid was formed.

### Methods 15 and 16: Preparation of Crystalline Compound A, n-propanesulfonic acid salt

### Method 15: Crystallisation of Amorphous Material

Amorphous Compound A, n-propanesulfonic acid salt (20 mg; see Method 14 above) was dissolved in iso-propanol (60 TL) and iso-propyl acetate (180 TL) was added. After three days crystalline needles were observed.

### Method 16: Reaction Crystallisation

Compound A (229 mg; see Preparation A above) was dissolved in iso-propanol (1.43 mL). n-Propanesulfonic acid was added (1 eq., 95%, 48 TL). Ethyl acetate was added (2 mL), and then the solution was seeded with crystalline salt from Method 15 above. Further ethyl acetate was added (5 mL) and the slurry was left overnight to crystallize. The crystals were filtered off, washed with ethyl acetate (3×0.3 mL) and dried under vacuum at 40° C.

Compound A, n-propanesulfonic acid salt was characterised by NMR as follows: 13 mg of the salt was dissolved in deuterated methanol (0.7 mL) troscopy. A combination of 1D (¹H, ¹³C) and 2D (gCOSY) NMR experiments were used. All data were in good agreement with the theoretical structure of the salt, shown below. The molecule exists in two conformations in methanol. Based on the integral of the peak assigned to H12 (dominant conformer) and peak assigned to H12′ (other conformer), the ratio between the two conformers was found to be 65:35. H22 could not be observed as these protons were in fast exchange with the solvent

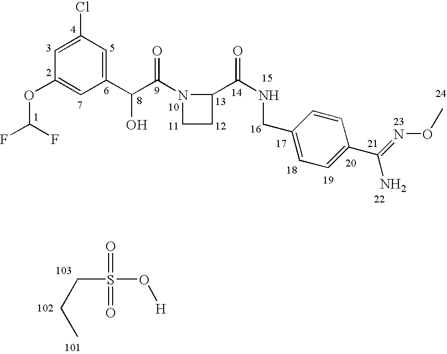

Both the proton and the carbon resonance corresponding to position 1 are split due to the spin-coupling with the two fluorine nuclei in that position. The coupling constants are ²J_{HF}=74 Hz and ¹J_{CF}=260 HZ.

¹H and ¹³C NMR chemical shift assignment and proton-proton correlations are shown in Table 6.

TABLE 6

| Atom | | | ¹H shift/ppm^{b} and | |
|---|---|---|---|---|
| No. | Type | ¹³C shift/ppm^{a} | multiplicity^{c} | J_{HH}/Hz |
| 1 | CH | 117.5^{e} | 6.89 (t) | 74 (²J_{HF}) |
| 1′ | | 117.5^{e} | 6.88 (t) | |
| 2 | C | 153.5 | | |
| 2′ | | 153.5 | | |
| 3 | CH | 120.0 | 7.16 (s) | |
| 3′ | | 119.7 | 7.13 (s) | |
| 4 | C | 136.2 | | |
| 4′ | | 135.9 | | |
| 5 | CH | 125.1 | 7.36 (s) | |
| 5′ | | 124.9 | 7.31 (s) | |
| 6 | C | 144.5 | | |
| 6′ | | 145.3 | | |
| 7 | CH | 117.3 | 7.20 (s) | |
| 7′ | | 117.2 | 7.16 (s) | |
| 8 | CH | 72.9 | 5.20 (s) | |
| 8′ | | 74.1 | 5.12 (s) | |
| 9 | CO | 173.1 | | |
| 9′ | | 173.8 | | |
| 11 | CH₂ | 51.6 | a: 4.37 (m) | |
| | | | b: 4.20 (m) | |
| 11′ | | 49.0 | a: 4.06 (m) | |
| | | | b: 3.98 (m) | |
| 12 | CH₂ | 21.7 | a: 2.53 (m) | |
| | | | b: 2.29 (m) | |
| 12′ | | 23.2 | a: 2.69 (m) | |
| | | | b: 2.15 (m) | |
| 13 | CH | 63.1 | 4.80 (m) | |
| 13′ | | 66.2 | 5.22 (m) | |
| 14 | CO | 172.9 | | |
| 14′ | | 173.8 | | |
| 15 | NH | | 8.75 (t, br) | 5.5 |
| 15′ | | | 8.79 (t, br) | 5.5 |
| 16 | CH₂ | 43.5 | 4.59 (AB-pattern) | 16.0 and 6.6 |
| | | | 4.45 (AB-pattern) | 16.0 and 5.3 |
| 16′ | | 43.6 | 4.51 | |
| | | | 4.50 | |
| 17 | C | 146.9 | | |
| 17′ | | 147.0 | | |
| 18 | CH | 129.1 | 7.54 (d) | 8.5 |
| 18′ | | 129.2 | 7.57 (d) | 8.5 |
| 19 | CH | 129.2 | 7.67 (d) | 8.5 |
| 19′ | | 129.4 | 7.69 (d) | 8.5 |
| 20 | C | 124.9 | — | |
| 20′ | | 124.9 | | |
| 21 | C | 162.4 | | |
| 21′ | | 162.4 | | |
| 22 | NH₂ | | Not observed | |
| 24 | CH₃ | 64.7 | 3.96 (s) | |
| 101 | CH | 13.7 | 1.0 (t) | |
| 102 | CH | 19.6 | 1.78 (m) | |
| 103 | CH | 54.6 | 2.75 (m) | |

^{a}Relative to the solvent resonance at 49.0 ppm.
^{b}Relative to the solvent resonance at 3.30 ppm.
^{c}s = singlet, t = triplet, m = multiplet, br = broad, d = doublet.
^{d}Obtained in the gCOSY experiment.
^{e}The resonance is a triplet due to coupling with the two fluorine nuclei.
¹J_{CF} = 260 Hz.

HRMS calculated for C₂₅H₃₁ClF₂N₄O₈S (M−H)⁻ 619.1441, found 619.1436.

Crystals of Compound A, n-propanesulfonic acid salt (obtained by way of one or more of Examples 15 and 16 above) were analyzed by XRPD and the results are tabulated below (Table 7) and are shown in FIG. 3.

TABLE 7

| d value (Å) | Intensity (%) | Intensity |
|---|---|---|
| 14.0 | 4 | w |
| 12.4 | 87 | vs |
| 10.0 | 30 | s |
| 8.0 | 3 | vw |
| 7.5 | 7 | m |
| 7.0 | 0.6 | vw |
| 6.7 | 1 | vw |
| 6.4 | 1 | vw |
| 6.2 | 12 | m |
| 6.1 | 3 | vw |
| 5.8 | 100 | vs |
| 5.7 | 11 | m |
| 5.5 | 3 | vw |
| 5.4 | 5 | w |
| 5.3 | 5 | w |
| 5.2 | 2 | vw |
| 5.1 | 3 | vw |
| 4.94 | 3 | vw |
| 4.78 | 21 | s |
| 4.68 | 42 | s |
| 4.51 | 10 | m |
| 4.49 | 7 | m |
| 4.40 | 5 | w |
| 4.32 | 10 | m |
| 4.29 | 10 | m |
| 4.25 | 22 | s |
| 4.19 | 14 | m |
| 4.14 | 15 | m |
| 4.07 | 23 | s |
| 4.04 | 20 | m |
| 3.94 | 16 | m |
| 3.88 | 10 | m |
| 3.73 | 15 | m |
| 3.65 | 2 | vw |
| 3.59 | 3 | vw |
| 3.48 | 18 | m |
| 3.28 | 23 | m |
| 3.12 | 4 | w |
| 3.06 | 3 | vw |
| 2.97 | 6 | w |
| 2.84 | 2 | vw |
| 2.81 | 3 | vw |
| 2.76 | 2 | vw |
| 2.73 | 3 | vw |
| 2.70 | 2 | vw |
| 2.57 | 2 | vw |
| 2.54 | 6 | w |
| 2.51 | 6 | w |
| 2.46 | 8 | m |
| 2.42 | 2 | vw |
| 2.39 | 3 | vw |
| 2.36 | 3 | vw |
| 2.32 | 2 | vw |
| 2.14 | 3 | vw |
| 2.01 | 2 | vw |

DSC showed an endotherm with an extrapolated melting onset temperature of ca. 135° C. TGA showed no decrease in mass around the melting point.

### Method 17

### Method 17-A: Preparation of amorphous Compound A n-butane sulfonic acid salt

Amorphous Compound A (277 mg) was dissolved in IPA (1.77 ml) and butane sulfonic acid (approx. 1 eq. 70 μL) was added. Ethyl acetate (6 ml) was added and the solvent was evaporated until dry, amorphous solid was formed.

### Method 17-B: Preparation of Crystalline Compound A Butane sulfonic acid salt

Amorphous Compound A butane sulfonic acid salt (71.5 mg; see preparation above) was slurried in ethyl acetate (500 μl) over night. The crystals were filtered off and were air-dried.

Compound A, butanesulfonic acid salt was charaterised by NMR as follows:

21.6 mg of the salt was dissolved in deuterated dimethylsulfoxide (0.7 ml) and was investigated with ¹H and ¹³C NMR spectroscopy.

The spectra are very similar to other salts of the same compound and in good agreement with the structure shown below. Most resonances in the spectra are present as sets of two peaks due to the slow rotation around the C9-N10 bond, which results in two atropisomers that simultaneously exist in the solution. This is shown for other salts of the same compound.

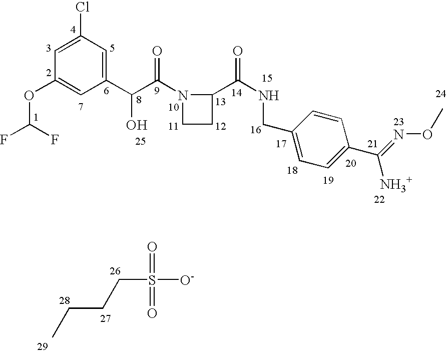

The two fluorine nuclei in position 1 give rise to split resonances for the proton and the carbon in that position. The coupling constants are ²J_{HF}=73 Hz and ¹J_{CF}=258 Hz. Chemical shifts for protons and carbons are presented in Table 1. Protons in position 22 and 24 are not detected due to chemical exchange. There is a very broad hump between 8 and 9 ppm in the proton spectrum corresponding to these protons.

TABLE 8
¹H and ¹³C NMR chemical shift assignment of Compound A
n-butanesulfonate salt in deuterated dimethylsulfoxide at 25° C.

| Atom | | ¹³C shift/ | ¹H shift/ppm^{b} and | |
|---|---|---|---|---|
| No. | Typ | ppm^{a} | multiplicity^{c} | J_{HH}/Hz |
| 1 | CHF₂ | 116.3^{d} | 7.29 (t) | 73 (²J_{HF}) |
| 1′ | | 116.3^{d} | 7.28 (t) | 73 (²J_{HF}) |
| 2 | C | 151.5 | na | na |
| 2′ | | 151.3 | na | na |
| 3 | CH | 118.0 | 7.25 (t)^{e} | nd |
| 3′ | | 117.6 | 7.21 (t)^{e} | nd |
| 4 | C | 133.8 | na | na |
| 4′ | | 133.4 | na | na |
| 5 | CH | 123.8 | 7.34 (t)^{e} | nd |
| 5′ | | 123.6 | 7.25 (t)^{e} | nd |
| 6 | C | 144.5 | na | na |
| 6′ | | 145.2 | na | na |
| 7 | CH | 116.3 | 7.19 (t)^{e} | nd |
| 7′ | | 116.1 | 7.12 (t)^{e} | nd |
| 8 | CH | 70.9 | 5.13 (s) | na |
| 8′ | | 71.2 | 4.99 (s) | na |
| 9 | CO | 170.6 | na | na |
| 9′ | | 171.1 | na | na |
| 11 | CH₂ | 50.0 | a: 4.24 (m) b: 4.12 (m) | nd |
| 11′ | | 46.9 | 3.85 (m) | nd |
| 12 | CH₂ | 20.5 | a: 2.41 (m) b: 2.10 (m) | nd |
| 12′ | | 21.7 | a: 2.60 (m) b: 2.02 (m) | nd |
| 13 | CH | 61.2 | 4.65 (dd) | 5.6 and 8.9 |
| 13′ | | 63.9 | 5.12 (m) | nd |
| 14 | CO | 170.2 | na | na |
| 14′ | | 171.0 | na | na |
| 16 | CH₂ | 41.8 | 4.38 (m) | nd |
| 16′ | | 42.0 | 4.38 (m) | nd |
| 17 | C | 144.7 | na | na |
| 18 | CH | 127.5 | 7.44 (d) | 8.2 |
| | | 127.6 | 7.44 | nd |
| 19 | CH | 127.8 | 7.66 (d) | 8.2 |
| 20 | C | 125.1 | na | na |
| 21 | C | 157.9 | na | na |
| 24 | CH₃ | 63.3 | 3.83 (s) | na |
| 24′ | | 63.3 | 3.82 (s) | na |
| 26 | CH₂ | 51.4 | 2.41 (m) | nd |
| 27 | CH₂ | 27.3 | 1.52 (m) | nd |
| 28 | CH₂ | 21.7 | 1.30 (m) | nd |
| 29 | CH₃ | 14.0 | 0.83 (t) | 7.3 |

^{a}Relative to the solvent resonance at 49.0 ppm.
^{b}Relative to the solvent resonance at 3.30 ppm.
^{c}s = singlet, d = doublet, dd = doublet of doublets, t = triplet, m = multiplet.
^{d}The resonance is a triplet due to coupling with the two fluorine nuclei F1.
¹J_{CF} = 258 Hz.
^{e}The ⁴J_{HH} coupling with the meta-protons is not fully resolved.
na = not applicable,
nd = not determined

HRMS calculated for C₂₆H₃₂ClF₂N₄O₈S (M−H)⁻ 633.1597, found 633.1600

Crystals of Compound A n-butanesulfonic acid salt (obtained as described above in Method 17-B) were analyzed by XRPD and the results are tabulated below (Table 9) and are shown in FIG. 4.

TABLE 9

| d-value (Å) | Intensity (%) | Intensity |
|---|---|---|
| 14.3 | 8 | m |
| 12.8 | 81 | vs |
| 10.3 | 44 | s |
| 8.2 | 4 | w |
| 7.7 | 13 | m |
| 6.7 | 2 | vw |
| 6.4 | 8 | m |
| 6.2 | 18 | m |
| 6.0 | 100 | vs |
| 5.8 | 29 | s |
| 5.6 | 4 | w |
| 5.4 | 11 | m |
| 5.3 | 16 | m |
| 5.1 | 15 | m |
| 4.98 | 6.5 | w |
| 4.91 | 34 | s |
| 4.76 | 56 | s |
| 4.57 | 20 | m |
| 4.42 | 13 | m |
| 4.36 | 19 | m |
| 4.30 | 45 | s |
| 4.18 | 42 | s |
| 4.13 | 88 | vs |
| 4.01 | 34 | s |
| 3.92 | 28 | s |
| 3.82 | 18 | m |
| 3.64 | 6.6 | w |
| 3.58 | 16 | m |
| 3.47 | 5 | w |
| 3.44 | 6 | w |
| 3.38 | 12 | m |
| 3.35 | 32 | s |
| 3.32 | 22 | s |
| 3.29 | 12 | m |
| 3.20 | 8 | m |
| 3.17 | 9 | m |
| 3.02 | 12 | m |
| 2.90 | 6 | w |
| 2.81 | 3.9 | vw |
| 2.75 | 3 | vw |
| 2.64 | 3.5 | vw |
| 2.59 | 10 | m |
| 2.57 | 8 | m |
| 2.50 | 4 | w |
| 2.45 | 5 | w |
| 2.40 | 6 | w |
| 2.31 | 3 | vw |

DSC showed an endotherm with an extrapolated melting onset temperature of ca 118° C. and TGA showed a 0.04% weight loss.

### Method 18: Preparation of Salts of Compound B

### Method 18-A: General Method for Salt Preparation

The following generic method was employed to prepare salts of Compound B: 200 mg of compound B (see Preparation B above) was dissolved in 5 mL of MIBK (methyl isobutyl ketone). To this solution was added a solution of the relevant acid (1.0 or 0.5 molar equivalent, as indicated in Table 10) dissolved in 1.0 mL of MIBK. After stirring for 10 minutes at room temperature, the solvent was removed by way of a rotary evaporator. The remaining solid material was re-dissolved in about 8 mL of acetonitrile:H₂O (1:1). Freeze-drying afforded colorless amorphous material in each case.

Acid Employed:

Esylate (ethanesulfonic acid)
Besylate (benzene sulfonic acid)
Cyclohexylsulphamate
Sulphate
Bromide
p-Toluenesulphonate
2-Naphtalenesulfonate
Hemisulfate
Methanesulphonate
Nitrate
Hydrochloride

Appropriate characterising data are shown in Table 10

TABLE 10

| Salt | Mw acid | Mw salt | MS ES- |
|---|---|---|---|
| Esylate | 110.13 | 643.01 | 108.8 |
| | | | 531.1 |
| | | | 641.0 |
| Besylate | 158.18 | 691.06 | 156.8 |
| | | | 531.1 |
| | | | 689.2 |
| Cyclohexylsulphamate | 179.24 | 712.12 | 177.9 |
| | | | 531.2 |
| | | | 710.4 |
| Sulphate | 98.08 | 630.96 | 531.1 |
| Bromide | 80.91 | 613.79 | 531.2 |
| | | | 613.1 |
| p-Toluenesulphonate | 172.20 | 705.08 | 170.9 |
| | | | 531.1 |
| | | | 703.1 |
| 2- | 208.24 | 741.12 | 206.9 |
| Naphtalenesulfonate | | | 531.1 |
| | | | 739.3 |
| Hemisulfate | 98.07 | 1163.8 | 531.1 |
| | | (1:2) | 631.0 |
| | | 630.85 | |
| | | (1:1) | |
| Methanesulphonate | 96.11 | 628.99 | 531.1 |
| | | | 627.1 |
| Nitrate | 63.01 | 595.89 | 531.0 |
| | | | 594.0 |
| Hydrochloride | 36.46 | 569.34 | 531.0 |
| | | | 569.0 |

All salts formed in this Example were amorphous.

### Method 18-B

Further amorphous salts of Compound B were made using analogous techniques to those described in Method 18-A above for the following acids:
1,2-Ethanedisulfonic (0.5 salt)
1S-Camphorsulfonic
(+/−)-Camphorsulfonic
p-Xylenesulfonic
2-Mesitylenesulfonic
Saccharin
Maleic
Phosphoric
D-glutamic
L-arginine
L-lysine
L-lysine*HCl

### Method 18-C: Preparation of Amorphous Compound B, Hemi-1,5-naphtalenedisulfonic acid salt

Amorphous Compound B (110.9 mg) was dissolved in 2.5 mL 2-propanol and 0.5 equivalent of 1,5-naphthalene-disulfonic acid tetrahydrate was added (dissolved in 1 mL 2-propanol). The sample was stirred overnight. Only small particles (amorphous) or oil drops were observed by microscopy. The sample was evaporated to dryness.

### Method 18-D: Preparation of Crystalline Compound B, hemi-1,5-naphtalenedisulfonic acid salt

The crystallization experiment was carried out at ambient temperature. Amorphous Compound B (0.4 gram) was dissolved in ethanol (1.5 mL) and 0.5 eq of 1,5-naphthalene-disulfonic acid tetrahydrate (1.35 gram, 10% in ethanol) was added. Heptane (0.7 mL) was then added until the solution became slightly cloudy. After about 15 minutes the solution became turbid. After about 30 minutes thin slurry was obtained and additional heptane (1.3 mL) was added. The slurry was than left overnight for ripening. To dilute the thick slurry, a mixture of ethanol and heptane (1.5 mL and 1.0 mL respectively) was added. After about 1 hour the slurry was filtered and the crystals were washed with a mixture of ethanol and heptane (1.5:1) and finally with pure heptane. The crystals were dried at ambient temperature in 1 day. The dry crystals weighed 0.395 g.

### Method 18-E: Preparation of Crystalline Compound B, hemi-1,5-naphtalenedisulfonic acid salt

Amorphous Compound B (1.009 gr) was dissolved in 20 mL 2-propanol+20 mL ethyl acetate. 351.7 mg 1,5-naphtalene-disulfonic acid tetrahydrate, dissolved in 20 mL 2-propanol, was added drop by drop. Precipitation occurred in about 5 minutes. The slurry was stirred over night and then filtered.

### Method 18-F: Preparation of Crystalline Compound B, hemi-1,5-naphtalenedisulfonic acid salt

430.7 mg of the 1,5-naphtalene-disulfonic acid salt was dissolved in 30 mL 1-propanol. The solution was heated to boiling in order to dissolve the substance. The solution was left over night at ambient temperature for crystallization and then the crystals were filtered off.

### Method 18-G: Preparation of Crystalline Compound B, hemi-1,5-naphtalenedisulfonic acid salt

The mother liquid from Method 18-F was evaporated and the solid rest (61.2 mg) was dissolved in 6 mL acetonitrile/1-propanol, ratio 2:1. The solution was left overnight at ambient temperature to crystallize and then the crystals were filtered off.

### Method 18-H: Preparation of Crystalline Compound B, hemi-1,5-naphtalenedisulfonic acid salt

The sample from Method 18-C was dissolved in about 2 mL methanol. Ethanol (about 3 mL) was added as anti-solvent at ambient temperature and seeds were added. No crystallization occurred, so solvents were evaporated (about half of the amount) and a new portion of ethanol (about 2 mL) and seeds were added. Crystalline particles were formed when stirred at ambient temperature during night.

### Method 18-I: Preparation of Crystalline Compound B, hemi-1,5-naphtalenedisulfonic acid salt

Amorphous Compound B (104.1 mg) was dissolved in 2-propanol and 1 equivalent of 1,5-naphthalene-disulfonic acid tetrahydrate, dissolved in 2-propanol, was added In total, the 2-propanol amount was about 2.5 mL. The solution was stirred at 44° C. for about 80 minutes and a precipitate was formed. The particles were crystalline according to polarised light microscopy. The sample was filtered.

### Method 18-J: Preparation of Crystalline Compound B, hemi-1,5-naphtalenedisulfonic acid salt

Compound B, hemi-1,5-naphtalenedisulfonic acid salt (56.4 mg) was dissolved in 1.5 mL methanol. Methyl ethyl ketone (3 mL) was added. Seeds were added to the solution and crystallization started. The crystals were filtered off, washed with methyl ethyl ketone and air dried.

### Method 18-K: Preparation of crystalline Compound B, hemi-1,5-naphtalenedisulfonic acid salt

Amorphous Compound B (161,0 mg) was dissolved in 3.5 mL 1-Butanol and the solution was heated to 40° C. In another beaker 57.4 mg of naphthalene-disulfonic acid tetrahydrate was dissolved in 3 mL 1-Butanol. A couple of drops of the acid solution were added to the solution of compound B. Then seeds were added to the solution and after 2 hours the rest of the acid solution was added (at 40° C.) slowly. Then the temperature was slowly decreased to room temperature and the experiment was left under stirring overnight. The slurry was filtered, washed with 1-Butanol and dried under vacuum at 44° C. for 2 hours. The yield was 83%.

Characterisation

Crystals of Compound B, hemi-1,5-naphtalenedisulfonic acid salt, obtained by way of Method 18-D above, was charaterised by NMR as follows:

21.3 mg of the salt was dissolved in deuterated methanol, 0.7 ml was investigated with NMR spectroscopy. A combination of 1D (¹H, ¹³C and selective NOE) and 2D (gCOSY, GHSQC and gHMBC) NMR experiments was used.

All data are in good agreement with the proposed structure, shown below. All carbons and the protons attached to carbons are assigned. Protons attached to heteroatoms are exchanged for deuterium from the solvent and are not detected. Most resonances in the 1D ¹H and ¹³C NMR spectra are present as sets of two peaks. The reason for this is a slow rotation around the C9-N10 bond, which results in two atropisomers that simultaneously exist in the solution. The 1D NOE experiment is an evidence for this. When a resonance of one atropisomer is irradiated, the saturation is transferred to the corresponding peak of the other atropisomer. The resonances corresponding to the 1,5-naphtalenedisulfonate counter ion do not show atropisomerism.

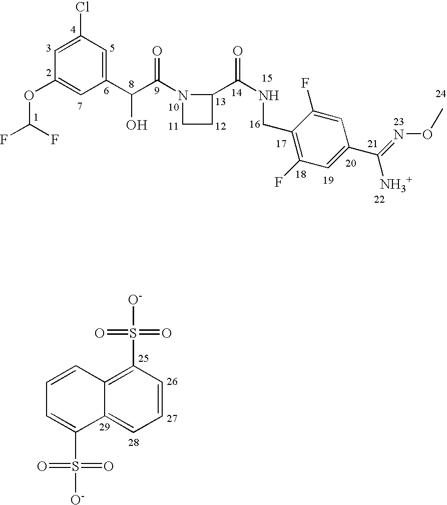

There are four fluorine atoms in the molecule. They give rise to split resonances for some protons and carbons. Both the proton and the carbon resonance corresponding to position 1 are split due to the spincoupling with the two fluorine nuclei in that position. The coupling constants are ²J_{HF}=73 Hz and ¹J_{CF}=263 Hz. Further, the proton resonance corresponding to H19 is a distorted doublet with ³J_{HF}=6.9 Hz due to the spincoupling with the fluorine nuclei in position 18. Carbon resonances corresponding to C17, C18, C19 and C20 also exhibit couplings with these fluorine nuclei. The C17 and C20 resonances are triplets with ²J_{CF}=19 Hz and ³J_{CF}=11 Hz, respectively. The C18 resonance is a doublet of doublets with coupling constants ¹J_{CF}=251 Hz and ³J_{CF}=8 Hz. The C19 resonance is a multiplet.

Comparing the magnitudes of integrals for resonances corresponding to the 1,5-naphtalenedisulfonate counter ion and the mother compound gives the stoichiometric relation of a single 1,5-naphtalenedisulfonate counter ion crystallized with two molecules of the mother compound.

¹H and ¹³C NMR chemical shift assignment and proton-proton correlations are shown in Table 11.

TABLE 11

| | | ¹³C | | | Through-bond |
|---|---|---|---|---|---|
| Atom | | shift/ | ¹H shift/ppm^{b} and | | correlation |
| No. | Typ | ppm^{a} | multiplicity^{c} | J_{HH}/Hz | to ¹H^{d} |
| 1 | CHF₂ | 117.5^{e} | 6.91 (t) | 73 (²J_{HF}) | nd |
| 1′ | | 117.5^{e} | 6.87 (t) | 73 (²J_{HF}) | nd |
| 2 | C | 153.5 | na | na | na |
| 2′ | | 153.3 | na | na | na |
| 3 | CH | 120.0 | 7.14 (t)ⁿ | nd | 5, 7 |
| 3′ | | 119.6 | 7.11 (t)ⁿ | nd | 5′, 7′ |
| 4 | C | 136.1 | na | na | na |
| 4′ | | 135.8 | na | na | na |
| 5 | CH | 125.0 | 7.31 (t)ⁿ | nd | 3, 7 |
| 5′ | | 124.9 | 7.28 (t)ⁿ | nd | 3′, 7′ |
| 6 | C | 144.4 | na | na | na |
| 6′ | | 145.3 | na | na | na |
| 7 | CH | 117.2 | 7.16 (t)ⁿ | nd | 3, 5 |
| 7′ | | 117.1 | 7.12 (t)ⁿ | nd | 3′, 5′ |
| 8 | CH | 72.9 | 5.15 (s) | na | nd |
| 8′ | | 73.6 | 5.07 (s) | na | nd |
| 9 | CO | 173.0 | na | na | na |
| 9′ | | 173.5 | na | na | na |
| 11 | CH₂ | 51.5 | a: 4.29 (m) b: 4.13 | nd | 12, 13 |
| 11′ | | 48.6 | (m) | nd | 12′, 13′ |
| | | | a: 4.01 (m) b: 3.93 | | |
| | | | (m) | | |
| 12 | CH₂ | 21.7 | a: 2.46 (m) b: 2.17 | nd | 11, 13 |
| 12′ | | 22.8 | (m) | nd | 11′, 13′ |
| | | | a: 2.61 (m) b: 2.03 | | |
| | | | (m) | | |
| 13 | CH | 62.8 | 4.70 (dd) | 6.0 and | 12 |
| 13′ | | 65.8 | 5.14 (dd) | 9.4 | 12′ |
| | | | | 5.6 and | |
| | | | | 9.1 | |
| 14 | CO | 172.4 | na | na | na |
| 14′ | | 173.2 | na | na | na |
| 16 | CH₂ | 32.3 | 4.51 (m) | nd | nd |
| 16′ | | 32.5 | 4.51 (m) | nd | nd |
| 17 | C | 121.0^{f} | na | na | na |
| 18 | CF | 162.8^{g} | na | na | na |
| 19 | CH | 112.7ⁱ | 7.35 (d) | 6.9 (³J_{HF}) | nd |
| 20 | C | 127.9^{k} | na | na | na |
| 21 | C | 160.0 | na | na | na |
| 21′ | | 159.9 | na | na | na |
| 24 | CH₃ | 64.8 | 3.93 (s) | na | nd |
| 24′ | | 64.8 | 3.92 (s) | na | nd |
| 25 | C | 142.4 | na | na | na |
| 26 | CH | 126.8 | 8.16 (d) | 7.2 | 27, 28 |
| 27 | CH | 125.9 | 7.54 (dd) | 8.6 and | 26, 28 |
| | | | | 7.2 | |
| 28 | CH | 131.0 | 8.97 (d) | 8.6 | 26, 27 |
| 29 | C | 131.1 | na | na | na |

^{a}Relative to the solvent resonance at 49.0 ppm.
^{b}Relative to the solvent resonance at 3.30 ppm.
^{c}s = singlet, d = doublet, dd = doublet of doublets, t = triplet, m = multiplet.
^{d}Obtained in the gCOSY experiment.
^{e}The resonance is a triplet due to coupling with the two fluorine nuclei F1.
¹J_{CF} = 263 Hz.
^{f}The resonance is a triplet due to coupling to the two fluorine nuclei F18.
²J_{CF} = 19 Hz.
^{g}The resonance is a doublet of doublets due to coupling to the two fluorine nuclei F18.
¹J_{CF} = 251 Hz and
³J_{CF} = 8 Hz.
ⁱThe resonance is a multiplet due to coupling to the two fluorine nuclei F18.
^{k}The resonance is a triplet due to coupling to the two fluorine nuclei F18.
³J_{CF} = 11 Hz.
ⁿThe ⁴J_{HH} coupling with the meta-protons is not fully resolved.
na = not applicable,
nd = not determined

Crystals of Compound B, hemi-1,5-naphtalenedisulfonic acid salt (obtained by way of Method 18-I above, were analyzed by XRPD and the results are tabulated below (Table 12) and are shown in FIG. 5.

TABLE 12

| | Intensity | |
|---|---|---|
| d value (Å) | (%) | Intensity |
| 18.3 | 99 | vs |
| 12.5 | 22 | s |
| 9.9 | 22 | s |
| 9.1 | 67 | vs |
| 8.0 | 18 | m |
| 7.5 | 17 | m |
| 6.8 | 37 | s |
| 6.7 | 59 | s |
| 6.1 | 39 | s |
| 6.0 | 21 | s |
| 5.6 | 66 | vs |
| 5.5 | 98 | vs |
| 4.94 | 48 | s |
| 4.56 | 59 | s |
| 4.39 | 35 | S |
| 4.27 | 33 | s |
| 4.13 | 81 | vs |
| 4.02 | 87 | vs |
| 3.86 | 88 | vs |
| 3.69 | 69 | vs |
| 3.63 | 100 | vs |
| 3.57 | 49 | s |
| 3.48 | 53 | s |
| 3.23 | 35 | s |
| 3.19 | 43 | s |
| 3.16 | 38 | s |

DSC showed an endotherm with an extrapolated melting onset temperature of ca 183° C. and TGA showed a 0.3% weight loss between 25-110° C.

Abbreviations

Ac=acetyl
APCI=atmospheric pressure chemical ionisation (in relation to MS)
API=atmospheric pressure ionisation (in relation to MS)
aq.=aqueous
Aze(& (S)-Aze)=(S)-azetidine-2-carboxylate (unless otherwise specified)
Boc=tert-butyloxycarbonyl
br=broad (in relation to NMR)
CI=chemical ionisation (in relation to MS)
d=day(s)
d=doublet (in relation to NMR)
DCC=dicyclohexyl carbodiimide
dd=doublet of doublets (in relation to NMR)
DIBAL-H=di-isobutylaluminium hydride
DIPEA=diisopropylethylamine
DMAP=4-(N,N-dimethyl amino) pyridine
DMF=N,N-dimethylformamide
DMSO=dimethylsulfoxide
DSC=differential scanning colorimetry
DVT=deep vein thrombosis
EDC=1-(3-dimethylaminopropyl)-3-ethylcarbodiimide hydrochloride
eq.=equivalents
ES=electrospray
ESI=electrospray interface
Et=ethyl
ether=diethyl ether
EtOAc=ethyl acetate
EtOH=ethanol
Et₂O=diethyl ether
HATU=O-(azabenzotriazol-1-yl)-N,N′,N′-tetramethyluronium hexafluorophosphate
HBTU=[N,N,N′,N′-tetramethyl-O-(benzotriazol-1-yl)uronium hexafluorophosphate]
HCl=hydrochloric acid, hydrogen chloride gas or hydrochloride salt (depending on context)
Hex=hexanes
HOAc=acetic acid
HPLC=high performance liquid chromatography
LC=liquid chromatography
m=multiplet (in relation to NMR)
Me=methyl
MeOH=methanol
min.=minute(s)
MS=mass spectroscopy
MTBE=methyl tert-butyl ether
NMR=nuclear magnetic resonance
OAc=acetate
Pab=para-amidinobenzylamino
H-Pab=para-amidinobenzylamine
Pd/C=palladium on carbon
Ph=phenyl
PyBOP=(benzotriazol-1-yloxy)tripyrrolidinophosphonium hexafluorophosphate
q=quartet (in relation to NMR)
QF=tetrabutylammonium fluoride
rt/RT=room temperature
s=singlet (in relation to NMR)
solutol=PEG 660 12-hydroxy stearate (a non-ionic surfactant)
t=triplet (in relation to NMR)
TBTU=[N,N,N′,N′-tetramethyl-O-(benzotriazol-1-yl)uronium tetrafluoroborate]
TEA=triethylamine
Teoc=2-(trimethylsilyl)ethoxycarbonyl
TEMPO=2,2,6,6-tetramethyl-1-piperidinyloxy free radical
TFA=trifluoroacetic acid
TGA=thermogravimetric analysis
THF=tetrahydrofuran
TLC=thin layer chromatography
UV=ultraviolet

Prefixes n-, s-, i-, t- and tert- have their usual meanings: normal, secondary, iso, and tertiary.

The invention is illustrated by way of the following Examples.

### EXAMPLE 1

| | | |
|---|---|---|
| Compound A | 30 | μmol |
| PEG 400/ethanol/water 50/5/45 (w/w) % | to 1 | mL |

A formulation was prepared by dissolving Compound A in PEG 400/ethanol/water 50/5/45 (w/w) % followed by gently stirring. This composition was given to dogs orally by gavage once daily for 5 days. The dose 150 μmol/kg gave maximum plasma concentrations in the range 118-254 μM (118-254 μmol/L) after the first dose and 186-286 μM (186-286 μmol/L) after the fifth dose.

### EXAMPLE 2

| | | |
|---|---|---|
| Compound A | 40 | μmol |
| PEG 400/ethanol/water 50/5/45 (w/w) % | to 1 | mL |

A formulation was prepared by dissolving Compound A in PEG 400/ethanol/water 50/5/45 (w/w) % followed by gently stirring. This composition was given to rats orally by gavage once daily for 5 days. The dose 400 μmol/kg gave maximum plasma concentrations in the range 3.17-6.91 μM (3.17-6.91 μmol/L) after the first dose and 3.01-10.5 μM (3.01-10.5 μmol/L) after the fifth dose.

### EXAMPLE 3

| | | |
|---|---|---|
| Compound A | 80 | μmol |
| PEG 400/ethanol/water 50/5/45 (w/w) % | to 1 | mL |

A formulation was prepared by dissolving Compound A in PEG 400/ethanol/water 50/5/45 (w/w) % followed by gently stirring. This composition was given to rats orally by gavage once daily for 5 days. The dose 800 μmol/kg gave maximum plasma concentrations in the range 7.00-23.9 μM (7.00-23.9 μmol/L) after the first dose and 10.3-32.8 μM (10.3-32.8 μmol/L) after the fifth dose.

### EXAMPLE 4

| | | |
|---|---|---|
| Compound A | 250 | μmol |
| PEG 400/ethanol/water 50/5/45 (w/w) % | to 1 | mL |

A formulation was prepared by dissolving Compound A in PEG 400/ethanol/water 50/5/45 (w/w) % followed by gently stirring. The solubility of Compound A is at least 1000 times higher in this vehicle compared to water alone.

### EXAMPLE 5

| | | |
|---|---|---|
| Compound A | 21 | μmol |
| PEG 400/ethanol/water 20/10/70 (w/w) % | to 1 | mL |

A formulation was prepared by dissolving Compound A in PEG 400/ethanol/water 20/10/70 (w/w) % followed by gently stirring. The solubility of Compound A is at least 100 times higher in this vehicle compared to water alone.

### EXAMPLE 6

| | | |
|---|---|---|
| Compound A | 51 | μmol |
| PEG 400/ethanol/water 20/10/70 (w/w) % | to 1 | mL |

The water contained 50 μmol/mL Tartaric Acid

A formulation was prepared by dissolving Compound A in acidified PEG 400/ethanol/water 20/10/70 (w/w) % that was followed by gently stirring. The pH of this solution was 3.6. The solubility of Compound A is at least 250 times higher in this vehicle compared to water alone.

### EXAMPLE 7

| | | |
|---|---|---|
| Compound A | 44 | μmol |
| PEG 400/ethanol/water 30/5/65 (w/w) % | to 1 | mL |

A formulation was prepared by dissolving Compound A in PEG 400/ethanol/water 30/5/65 (w/w) % followed by gently stirring. The solubility of Compound A is at least 200 times higher in this vehicle compared to water alone.

### EXAMPLE 8

| | | |
|---|---|---|
| Compound A | 88 | μmol |
| PEG 400/ethanol/water 30/5/65 (w/w) % | to 1 | mL |

| | |
|---|---|
| The water contained 50 μmol/mL Tartaric Acid | q.s. |
| HCl to pH 3.6 | |

A formulation was prepared by dissolving Compound A in acidified PEG 400/ethanol/water 30/5/65 (w/w) % followed by gently stirring. The pH of this solution was set to 3.6 by addition of HCl. The solubility of Compound A is at least 400 times higher in this vehicle compared to water alone.

### EXAMPLE 9

| | | |
|---|---|---|
| Compound A | 120 | μmol |
| PEG 400/ethanol/water 40/5/55 (w/w) % | to 1 | mL |

A formulation was prepared by dissolving Compound A in PEG 400/ethanol/water 40/5/55 (w/w) % followed by gently stirring. The solubility of Compound A is at least 600 times higher in this vehicle compared to water alone.

### EXAMPLE 10

| | | |
|---|---|---|
| Compound A | 198 | μmol |
| PEG 400/ethanol/water 40/5/55 (w/w) % | to 1 | mL |

| | |
|---|---|
| The water contained 50 μmol/mL Tartaric Acid | q.s. |
| HCl to pH 3.8 | |

A formulation was prepared by dissolving Compound A in acidified PEG 400/ethanol/water 40/5/55 (w/w) % followed by gently stirring. The pH of this solution was set to 3.8 by addition of HCl. The solubility of Compound A is at least 1000 times higher in this vehicle compared to water alone. Formulations of Compound A in this vehicle are stable for at least 3 months at <−15° C.

### EXAMPLE 11

| | | |
|---|---|---|
| Compound A | 136 | μmol |
| Hydroxypropyl-β-cyclodextrin/water 40/60 (w/w) % | to 1 | mL |

| | |
|---|---|
| HCl to pH 3.7 | q.s. |

A formulation was prepared by dissolving Compound A in Hydroxypropyl-β-cyclodextrin/water 40/60 (w/w) % followed by gently stirring. The pH of this solution was set to 4.7 by addition of HCl. The solubility of Compound A is at least 700 times higher in this vehicle compared to water alone.

### EXAMPLE 12

| | | |
|---|---|---|
| Compound A | 76 | μmol |
| Hydroxypropyl-β-cyclodextrin/water 28/72 (w/w) % | to 1 | mL |

A formulation was prepared by dissolving Compound A in Hydroxypropyl-β-cyclodextrin/water 28/72 (w/w) % followed by gently stirring. The solubility of Compound A is at least 400 times higher in this vehicle compared to water alone.

### EXAMPLE 13

| | | |
|---|---|---|
| Compound A | 40 | μmol |
| PEG 400/ethanol/solutol ™/water 50/5/5/40 (w/w) % | to 1 | mL |

A formulation was prepared by dissolving Compound A in PEG 400/ethanol/solutol™/water 50/5/5/40 (w/w) % followed by gently stirring. The solubility of Compound A is at least 80 times higher in this vehicle compared to water alone.

### EXAMPLE 14

| | | |
|---|---|---|
| Compound A | 40 | μmol |
| PEG 400/water 40/60 (w/w) % | to 1 | mL |

A formulation was prepared by dissolving Compound A in PEG 400 followed by gently stirring for at least 1 hour, thereafter water was added to the final volume. The solubility of Compound A is at least 200 times higher in this vehicle compared to water alone.

### EXAMPLE 15

| | | |
|---|---|---|
| Compound A | 52 | μmol |
| PEG 400/water 35/65 (w/w) % | to 1 | mL |
| The water contained 50 μmol/mL Tartaric Acid | | |

A formulation was prepared by dissolving Compound A in PEG 400 followed by gently stirring for at least 1 hour, thereafter water was added to the final volume. The solubility of Compound A is at least 250 times higher in this vehicle compared to water alone.

### EXAMPLE 16

| | | |
|---|---|---|
| Compound A | 58 | μmol |
| PEG 400/water 50/50 (w/w) % | to 1 | mL |

A formulation was prepared by dissolving Compound A in PEG 400 followed by gently stirring for at least 1 hour, thereafter water was added to the final volume. The solubility of Compound A is at least 300 times higher in this vehicle compared to water alone.

### EXAMPLE 17

| | | |
|---|---|---|
| Compound A | 88 | μmol |
| PEG 400/water 67/33 (w/w) % | to 1 | mL |

A formulation was prepared by dissolving Compound A in PEG 400 followed by gently stirring for at least 1 hour, thereafter water was added to the final volume. The solubility of Compound A is at least 400 times higher in this vehicle compared to water alone.

### EXAMPLE 18

| | | |
|---|---|---|
| Compound A | 92 | μmol |
| PEG 400/ethanol/water 45/1/54 (w/w) % | to 1 | mL |

A formulation was prepared by dissolving Compound A in PEG 400/ethanol/water 45/1/54 (w/w) % followed by gently stirring. The solubility of Compound A is at least 450 times higher in this vehicle compared to water alone.

### EXAMPLE 19

| | | |
|---|---|---|
| Compound A | 159 | μmol |
| PEG 400/ethanol/water 45/1/54 (w/w) % | to 1 | mL |

| | |
|---|---|
| The water contained 50 μmol/mL Tartaric Acid | q.s. |
| HCl to pH 4.2 | |

A formulation was prepared by dissolving Compound A in acidified PEG 400/ethanol/water 45/1/54 (w/w) % followed by gently stirring. The pH of this solution was set to 4.2 with HCl. The solubility of Compound A is at least 800 times higher in this vehicle compared to water alone.

### EXAMPLE 20

| | | |
|---|---|---|
| Compound A | 101 | μmol |
| PEG 400/ethanol/water 45/2/53 (w/w) % | to 1 | mL |

A formulation was prepared by dissolving Compound A in PEG 400/ethanol/water 45/2/53 (w/w) % followed by gently stirring. The solubility of Compound A is at least 500 times higher in this vehicle compared to water alone.

### EXAMPLE 21

| | | |
|---|---|---|
| Compound A | 167 | μmol |
| PEG 400/ethanol/water 45/2/53 (w/w) % | to 1 | mL |

| | |
|---|---|
| The water contained 50 μmol/mL Tartaric Acid | q.s. |
| HCl to pH 4.3 | |

A formulation was prepared by dissolving Compound A in acidified PEG 400/ethanol/water 45/2/53 (w/w) % followed by gently stirring. The pH of this solution was set to 4.3 by addition of HCl. The solubility of Compound A is at least 800 times higher in this vehicle compared to water alone.

### EXAMPLE 22

| | | |
|---|---|---|
| Compound A | 46 | μmol |
| DMA/water 50/50 (w/w) % | to 1 | mL |

A formulation was prepared by dissolving Compound A in the vehicle followed by gently stirring for at least 1 hour. The solubility of Compound A is at least 230 times higher in this vehicle compared to water alone.

### EXAMPLE 23

| | | |
|---|---|---|
| Compound A | 29 | μmol |
| DMA/water 25/75 (w/w) % | to 1 | mL |

A formulation was prepared by dissolving Compound A in the vehicle followed by gently stirring for at least 1 hour. The solubility of Compound A is at least 150 times higher in this vehicle compared to water alone.

### EXAMPLE 24

| | | |
|---|---|---|
| Compound A | 5 | μmol |
| HCl | 10 | μmol |
| Water | to 1 | mL |

| | |
|---|---|
| HCl/NaOH to pH 3.6 | q.s. |

A formulation was prepared by dissolving Compound A in a lower volume of the double equimolar amount of HCl followed by gently stirring and dilution to 1 mL. The pH of the final solution was adjusted to 3.6. The solubility of Compound A is at least 20 times higher in this vehicle compared to water alone.

### EXAMPLE 25

| | | |
|---|---|---|
| Compound A | 10 | μmol |
| Water | to 1 | mL |

| | |
|---|---|
| HCl to pH 1.0 | q.s. |
| NaOH to pH 3.0 | q.s. |

A formulation was prepared by dissolving Compound A water and HCl was added to give pH 1 thereafter the solution was gently stirred. The pH of the final solution was adjusted to 3.0 with NaOH. The solubility of Compound A is at least 40 times higher in this vehicle compared to water alone. This formulation was given p.o to rats in a kinetic comparative study.

### EXAMPLE 26

| | | |
|---|---|---|
| Compound A | 100 | μmol |
| Miglyol | 0.25 | g/g Compound A |
| DMA | to | 1 mL |

A formulation was prepared by dissolving Compound A in 1 mL DMA/miglyol followed by gently stirring. The solubility of Compound A is at least 4000 times higher in this vehicle compared to water alone.

### EXAMPLE 27

| | | |
|---|---|---|
| Compound A | 100 | μmol |
| Miglyol | 0.25 | g/g Compound A |
| Ethanol | to 1 | mL |

A formulation was prepared by dissolving Compound A in 1 mL Ethanol/Miglyol followed by gently stirring. The solubility of Compound A is at least 4000 times higher in this vehicle compared to water alone.

### EXAMPLE 28

| | | |
|---|---|---|
| Compound A | 130 | μmol |
| Ethanol | to 1 | mL |

A formulation was prepared by dissolving Compound A in 1 mL ethanol followed by gently stirring. The substance is stable in this formulation more than 1 week.

### EXAMPLE 29

In order to prepare nanoparticles a stock solution of Compound A of about 100 mM in ethanol was used. Included was also 25% (w/w) Miglyol, calculated on the amount of the substance. The solutions were diluted 1/10 with the stabilizer solution, consisting of 0.2% (w/w) PVP and 0.25 mM SDS in water. The mixing, which is considered as a critical parameter during the nanoparticle preparation, was rapid and instant. The drug solution was rapidly injected into the stabilizer solution during ultrasonication. After the 1/10 dilution in the aqeous solution, nanoparticles of about 150 nm were achieved. After 6 hours at room temperature, the particle sizes were unchanged.

### EXAMPLE 30

| | | |
|---|---|---|
| Compound A | 4 | μmol |
| saline/ethanol/solutol 90/5/5 (w/w) % | to 1 | mL |

A formulation was prepared by dissolving Compound A in saline/ethanol/solutol 90/5/5 (w/w) % followed by gently stirring. The solution was given orally to rats and the plasma concentration of Compound D was 0.56 μmol/L after 1 hour. The solution was given subcutaneously to rats and the plasma concentrations of Compound D and A were 0.24 μmol/L and 0.6 μmol/L, respectively, after 1 hour.

### EXAMPLE 31

| | | |
|---|---|---|
| Compound B | 4 | μmol |
| saline/ethanol/solutol 90/5/5 (w/w) % | to 1 | mL |

A formulation was prepared by dissolving Compound B in saline/ethanol/solutol 90/5/5 (w/w) % followed by gently stirring. The solution was given orally to rats and the plasma concentrations of Compound B and Compound E were respectively 0.07 μmol/L and 0.65 μmol/L, after 1 hour. The solution was given subcutaneously to rats and the plasma concentrations of Compound B and E were 0.4 μmol/L and 0.3 μmol/L, respectively, after 1 hour.

### EXAMPLE 32

| | | |
|---|---|---|
| Compound C | 4 | μmol |
| saline/ethanol/solutol 90/5/5 (w/w) % | to 1 | mL |

A formulation was prepared by dissolving Compound C in saline/ethanol/solutol 90/5/5 (w/w) % followed by gently stirring. The solution was given orally to rats and the plasma concentrations of Compounds C and F were respectively 0.2 μmol/L and 0.5 μmol/L after 1 hour. The solution was given subcutaneously to rats and the plasma concentrations of Compounds C and F were 0.35 μmol/L and 0.5 μmol/L, respectively, after 1 hour.

### EXAMPLE 33

| | | |
|---|---|---|
| Compound D (trifluoroacetate salt) | 5 | μmol |
| Saline 9 mg/ml | to 1 | mL |

A formulation was prepared by dissolving the salt of Compound D in 1 mL saline followed by gently stirring.

### EXAMPLE 34

| | | |
|---|---|---|
| Compound D (trifluoroacetate salt) | 75 | μmol |
| EtOH | 0.05 | mL |
| Saline(9 mg/ml) | to 1 | mL |

A formulation was prepared by dissolving the salt of Compound D in 1 mL saline/ethanol solution followed by gently stirring.

### EXAMPLE 35

| | | |
|---|---|---|
| Compound D (trifluoroacetate salt) | 4 | μmol |
| EtOH | 0.02 | mL |
| saline | to 1 | mL |

A formulation was prepared by dissolving the salt of Compound D in 1 mL saline/etanol solution followed by gently stirring. The solution was given subcutaneously to rats and the plasma concentration of Compound D was 0.55 μmol/L after 1 hour.

### EXAMPLE 36

| | | |
|---|---|---|
| Compound E (acetate salt) | 4 | μmol |
| EtOH | 0.02 | mL |
| saline | to 1 | mL |

A formulation was prepared by dissolving the salt of Compound E in 1 mL saline/ethanol solution followed by gently stirring. The solution was given subcutaneously to rats and the plasma concentration of Compound E was 0.75 μmol/L after 1 hour.

### EXAMPLE 37

| | | |
|---|---|---|
| Compound F (trifluoroacetate salt) | 4 | μmol |
| EtOH | 0.02 | mL |
| saline | to 1 | mL |

A formulation was prepared by dissolving the salt of Compound F in 1 mL saline/ethanol solution followed by gently stirring. The solution was given subcutaneously to rats and the plasma concentration Compound F was 0.92 μmol/L after 1 hour.

### EXAMPLE 38

| | | |
|---|---|---|
| Compound E (acetate salt) | 22 | mg |
| Saline 9 mg/ml | to 1 | mL |

A formulation was prepared by dissolving the salt of Compound E in 1 mL saline followed by gently stirring.

### EXAMPLE 39

| | | |
|---|---|---|
| Compound F (trifluoroacetate salt) | 22 | mg |
| Saline 9 mg/ml | to 1 | mL |

A formulation was prepared by dissolving the salt of Compound F in 1 mL saline followed by gently stirring.

### EXAMPLE 40

| | | |
|---|---|---|
| Compound A (as esylate salt) | 14 | mg |
| water | to 1 | mL |

A solution was prepared by dissolving excess of Compound A as esylate salt in 3 mL water followed by gently stirring over night. A final concentration of the solution after filtration was monitored to 14 mg/ml at a pH of 2.7.

### EXAMPLE 41

| | | |
|---|---|---|
| Compound A (as esylate salt) | 33 | mg |
| Sodium phosphate buffer pH = 3.1 I = 0.1 | to 1 | mL |

A solution was prepared by dissolving 112 mg of Compound A as esylate salt in 3 mL sodium phosphate buffer followed by gently stirring over night. A final concentration of the solution after filtration was monitored to 33 mg/ml at a pH of 2.7.

### EXAMPLE 42

| | | |
|---|---|---|
| Compound A (as esylate salt) | 1.6 | mg |
| Sodium phosphate buffer pH = 6.9 I = 0.1 | to 1 | mL |

A solution was prepared by dissolving 20 mg of Compound A as esylate salt in 3 mL sodium phosphate buffer followed by gently stirring over night. A final concentration of the solution after filtration was monitored to 1.6 mg/ml at a pH of 6.5.

### EXAMPLE 43

The following freeze dried formulations can be made in accordance with techniques described in one or more of Examples 1-29 above:

| a. | | |
|---|---|---|
| Compound A | 10 | μmol |
| Mannitol | 10 | mg |
| Water | to 1 | mL |
| HCl to pH 1.0 | q.s. | |
| NaOH to pH 3.0 | q.s. | |
| b. | | |
| Compound D | 10 | μmol |
| Mannitol | 10 | mg |
| Water | to 1 | mL |
| HCl to pH 1.0 | q.s. | |
| NaOH to pH 3.0 | q.s. | |
| c. | | |
| Compound E | 10 | μmol |
| Mannitol | 10 | mg |
| Water | to 1 | mL |
| HCl to pH 1.0 | q.s. | |
| NaOH to pH 3.0 | q.s. | |
| d. | | |
| Compound F | 10 | μmol |
| Mannitol | 10 | mg |
| Water | to 1 | mL |
| HCl to pH 1.0 | q.s. | |
| NaOH to pH 3.0 | q.s. | |
| e. | | |
| Compound B | 10 | μmol |
| Mannitol | 10 | mg |
| Water | to 1 | mL |
| HCl to pH 1.0 | q.s. | |
| NaOH to pH 3.0 | q.s. | |
| f. | | |
| Compound C | 10 | μmol |
| Mannitol | 10 | mg |
| Water | to 1 | mL |
| HCl to pH 1.0 | q.s. | |
| NaOH to pH 3.0 | q.s. | |
| g. | | |
| Compound A (as esylate salt) | 14 | mg |
| Mannitol | 10 | mg |
| Water | to 1 | mL |
| HCl to pH 1.0 | q.s. | |
| NaOH to pH 3.0 | q.s. | |
| h. | | |
| Compound A (as besylate salt) | 14 | mg |
| Mannitol | 10 | mg |
| Water | to 1 | mL |

| | |
|---|---|
| HCl to pH 1.0 | q.s. |
| NaOH to pH 3.0 | q.s. |

The solutions are optionally sterile filtered, for example through a 0.22 μm membrane filter. Solutions (sterile or otherwise) are filled into appropriate vessels (e.g. vials) and the formulations are freeze-died using standard equipment. Vials may be sealed in freeze-dryer equipment under a nitrogen atmosphere.

### EXAMPLE 44

| | Weight | Amount |
|---|---|---|
| Compound A | 48 mg | 17% |
| Polyvinyl pyrrolidone K90 | 8 mg | 3% |
| Mannitol | 21 mg | 7% |
| Microcrystalline cellulose | 187 mg | 65% |
| Sodium starch glycolate | 21 mg | 7% |
| Sodium stearyl fumarate | 3 mg | 1% |

The excipients and drug were mixed and granulated with polyvinyl pyrrolidone K90 dissolved in water. The granules were then dried in a drying oven. The granulate was lubricated with sodiumstearylfumarate and compressed into tablets using an excenterpress.

Three individual tablets were tested for drug release in 900 ml media using a USP dissolution apparatus 2 (paddle+basket¹) at 50 rpm and 37° C. The dissolution media used were 0.1 M hydrochloric acid (pH 1) and 0.1 M sodium phosphate buffer (pH 6.8). In-line quantitation was performed using the C Technologies fibre optic system with 220 nm as the analytical wavelength when 0.1 M HCl was used as the dissolution media and with 260 nm as the analytical wavelength when phosphate buffer pH 6.8 was used as the dissolution media. 350 nm was used as the reference wavelength with both media. For the first two hours of the analysis the release value was measured every 15 minutes, and then every hour for the remainder of the analysis. The results are presented in the table below.
[¹ A custom made quadrangular basket of mesh wire, soldered in one of its upper, narrow sides to the end of a steel rod. The rod is brought through the cover of the dissolution vessel and fixed by means of two Teflon nuts, 3.2 cm from the centre of the vessel. The lower edge of the bottom of the basket is adjusted to be 1 cm above the paddle. The basket is directed along the flow stream with the tablet under test standing on its edge].

| | % released in | % released in |
|---|---|---|
| Time (min) | buffer pH 1.1 | buffer pH 6.8 |
| 0 | 0 | 0 |
| 15 | 100 | 44 |
| 30 | 100 | 49 |
| 45 | 100 | 51 |
| 60 | 100 | 53 |
| 120 | 100 | 57 |
| 180 | 100 | 61 |
| 240 | 100 | 63 |
| 360 | 100 | 67 |
| 480 | 100 | 70 |
| 600 | 100 | 75 |
| 720 | 100 | 77 |
| 840 | 100 | 79 |
| 960 | 100 | 82 |
| 1080 | 100 | 83 |
| 1200 | 100 | 86 |

### EXAMPLE 45

| | Weight | Amount |
|---|---|---|
| Esylate salt of Compound A | 58 mg | 20% |
| Polyvinyl pyrrolidone K90 | 8 mg | 3% |
| Mannitol | 21 mg | 7% |
| Microcrystalline cellulose | 177 mg | 62% |
| Sodium starch glycolate | 21 mg | 7% |
| Sodium stearyl fumarate | 3 mg | 1% |

The excipients and drug were mixed and granulated with polyvinyl pyrrolidone K90 dissolved in water. The granules were then dried in a drying oven. The granulate was lubricated with sodium stearyl fumarate and compressed into tablets using an excenterpress.

### EXAMPLE 46

| | Weight | Amount |
|---|---|---|
| Compound B | 48 mg | 17% |
| Polyvinyl pyrrolidone K90 | 8 mg | 3% |
| Mannitol | 21 mg | 7% |
| Microcrystalline cellulose | 187 mg | 65% |
| Sodium starch glycolate | 21 mg | 7% |
| Sodium stearyl fumarate | 3 mg | 1% |

The excipients and drug were mixed and granulated with polyvinyl pyrrolidone K90 dissolved in water. The granules were then dried in a drying oven. The granulate was lubricated with sodium stearyl fumarate and compressed into tablets using an excenterpress

### EXAMPLE 47

| | Weight | Amount |
|---|---|---|
| Compound C | 48 mg | 17% |
| Polyvinyl pyrrolidone K90 | 8 mg | 3% |
| Mannitol | 21 mg | 7% |
| Microcrystalline cellulose | 187 mg | 65% |
| Sodium starch glycolate | 21 mg | 7% |
| Sodium stearyl fumarate | 3 mg | 1% |

The excipients and drug were mixed and granulated with polyvinyl pyrrolicdone K90 dissolved in water. The granules were then dried in a drying oven. The granulate was lubricated with sodium stearyl fumarate and compressed into tablets using an excenterpress

### EXAMPLE 48

| | | |
|---|---|---|
| Compound A | 16 | μmol |
| PEG 414 | to 1 | mL |

A formulation was prepared by dissolving Compound A in acidified PEG414 followed by gently stirring.

### EXAMPLE 49

| | | |
|---|---|---|
| Compound A | 16 | μmol |
| PEG 300 | to 1 | mL |

A formulation was prepared by dissolving Compound A in acidified PEG300 followed by gently stirring.

### EXAMPLE 50

| | | |
|---|---|---|
| Compound A | 16 | μmol |
| PEG 200 | to 1 | mL |

A formulation was prepared by dissolving Compound A in acidified PEG200 followed by gently stirring.

### EXAMPLE 51

| | | |
|---|---|---|
| Compound G | 4 | μmol |
| saline/ethanol/solutol 90/5/5 (w/w) % | to 1 | mL |

A formulation was prepared by dissolving Compound G in saline/ethanol/solutol 90/5/5 (w/w) % followed by gently stirring.

### EXAMPLE 52

| | | |
|---|---|---|
| Compound J | 4 | μmol |
| saline/ethanol/solutol 90/5/5 (w/w) % | to 1 | mL |

A formulation was prepared by dissolving Compound J in saline/ethanol/solutol 90/5/5 (w/w) % followed by gently stirring.

### EXAMPLE 53

| | | |
|---|---|---|
| Compound H | 4 | μmol |
| saline/ethanol/solutol 90/5/5 (w/w) % | to 1 | mL |

A formulation was prepared by dissolving Compound H in saline/ethanol/solutol 90/5/5 (w/w) % followed by gently stirring.

### EXAMPLE 54

| | Weight | Amount |
|---|---|---|
| Compound A esylate salt | 500 mg | 66% |
| Polyvinyl pyrrolidone K30 | 100 mg | 13% |
| Microcrystalline cellulose | 100 mg | 13% |
| Crosslinked sodium CMC | 50 mg | 7% |
| Magnesium stearate | 5 mg | 1% |

Formulation can be prepared in accordance with Example 47 above.

### EXAMPLE 55

| | Weight | Amount |
|---|---|---|
| Compound A n-propane | 100 mg | 23% |
| sulphonic acid salt | | |
| Polyvinyl pyrrolidone K30 | 60 mg | 14% |
| Lactose monohydrate | 100 mg | 23% |
| Microcrystalline cellulose | 150 mg | 34% |
| Polyvinyl pyrrolidone | 20 mg | 5% |
| crosslinked | | |
| Sodium stearyl fumarate | 10 mg | 2% |

Formulation can be prepared in accordance with Example 47 above.

### EXAMPLE 56

| | Weight | Amount |
|---|---|---|
| Compound A besylate salt | 20 mg | 8% |
| Hydroxypropyl cellulose | 15 mg | 6% |
| Microcrystalline cellulose | 200 mg | 79% |
| Crosslinked sodium CMC | 15 mg | 6% |
| Sodium stearyl fumarate | 3 mg | 1% |

Formulation can be prepared in accordance with Example 47 above.

### EXAMPLE 57

| | | |
|---|---|---|
| Compound A | 24 | μmol |
| PEG 400/ethanol/water 25/10/65 (w/w) % | to 1 | mL |

A formulation was prepared by dissolving Compound A in PEG 400/ethanol/water 25/10/65 (w/w) % followed by gently stirring. The solubility of Compound A is at least 100 times higher in this vehicle compared to water alone. The formulation is stable in a freezer for at least 2 months.

### EXAMPLE 58

| | | |
|---|---|---|
| Compound A | 800 | μmol |
| PEG 400/ethanol/water 50/10/40 (w/w) % | to 1 | mL |

A formulation was prepared by dissolving Compound A in PEG 400/ethanol/water 50/10/40 (w/w) % followed by gently stirring. The solubility of Compound A is at least 2000 times higher in this vehicle compared to water alone.

### EXAMPLE 59

| Compound A | 500 | μmol |
|---|---|---|
| Citric acid | 200 | μmol |
| HCl to pH 3.6 | q.s. | |
| PEG 400/ethanol/9 mg/ml NaCl 40/10/50 (w/w) % | to 1 | mL |

A formulation was prepared by dissolving Compound A in PEG 400/ethanol/water 40/10/50 (w/w) % followed by gently stirring. The solubility of Compound A is at least 1500 times higher in this vehicle compared to water alone.

### EXAMPLE 60

| Compound A | 24 | μmol |
|---|---|---|
| citric acid | 5 | μmol |
| HCl to pH 3.2 | q.s. | |
| ethanol/water 12/88 (w/w) % | to 1 | mL |

A formulation was prepared by dissolving Compound A in ethanol followed by gently stirring, thereafter citric acid and water was added to final volume and the pH was set to 3.2. The solubility of Compound A is at least 100 times higher in this vehicle compared to water alone. The formulation is stable in a freezer for at least 1 month.

### EXAMPLE 61

| Compound A | 2 | μmol |
|---|---|---|
| citric acid | 5 | μmol |
| HCl to pH 3.6 | q.s. | |
| 9 mg/ml NaCl | to 1 | mL |

A formulation was prepared by dissolving Compound A and citric acid in physicological saline followed by gently stirring. The pH was set to 3.6. The formulation is stable in a freezer for at least 3 months.

### EXAMPLE 62

| Compound A (as besylate salt) | 140 | μmol |
|---|---|---|
| citric acid | 5 | μmol |
| HCl to pH 3.6 | q.s. | |
| PEG 400/ethanol/water 40/5/55 (w/w) % | to 1 | mL |

A formulation was prepared by dissolving Compound A in PEG 400/ethanol/water 40/5/55 (w/w) % containing citric acid followed by gently stirring and setting the pH to 3.6. The formulation is stable in a freezer for at least 1 month.

### EXAMPLE 63

| Compound A (as besylate salt) | 65 | μmol |
|---|---|---|
| citric acid | 5 | μmol |
| HCl to pH 3.3 | q.s. | |
| PEG 400/ethanol/water 20/5/75 (w/w) % | to 1 | mL |

A formulation was prepared by dissolving Compound A in PEG 400/ethanol/water 20/5/75 (w/w) % containing citric acid followed by gently stirring and the pH was set to 3.2.

### EXAMPLE 64

| | | |
|---|---|---|
| Compound D (as acetate salt) | 25 | μmol |
| PEG 400/ethanol/water 40/5/55 (w/w) % | to 1 | mL |

| | |
|---|---|
| Tartaric Acid: Component A (acetate salt of D) | q.s. |
| equimolar amount plus 5 mM excess | |
| HCl to pH 3.6 | |

A formulation was prepared by dissolving Compound D in acidified PEG 400/ethanol/water 40/5/55 (w/w) % followed by gently stirring. The pH of this solution was set to 3.6 by addition of HCl. Formulations of D in this vehicle are stable for at least 2 months at <−15° C.

### EXAMPLE 65

| | | |
|---|---|---|
| Compound A | 50 | mg |
| HPMC (15000 Cps) | 5 | mg |
| Solutol HS15 | 20 | mg |
| Water | to 1 | mL |

The HPMC was suspended in hot water and melted Solutol was added during vigourous stirring. This solution was chilled and Compound A was added under vigourous stirring to form a well dispersed suspension.

### EXAMPLE 66

| | | |
|---|---|---|
| Compound A (as besylate salt) | 50 | mg |
| HPMC (15000 Cps) | 5 | mg |
| Solutol HS15 | 20 | mg |
| Water | to 1 | mL |

The HPMC was suspended in hot water and melted Solutol was added during vigourous stirring. This solution was shilled and Compound A (besylate) was added under vigourous stirring to form a well dispersed suspension.

### EXAMPLE 67

| Compound D (as acetate salt) | 2 | μmol |
|---|---|---|
| citric acid | 5 | μmol |
| HCl to pH 3.6 | q.s. | |
| 9 mg/ml NaCl | to 1 | mL |

A formulation was prepared by dissolving Compound A and citric acid in physicological saline and stirring gently. The pH was set to 3.6. The formulation is stable in a freezer for at least 3 months.

### EXAMPLE 68

To prepare nanoparticles a stock solution of Compound B of about 100 mM in ethanol was used. Included was also 25% (w/w) Miglyol, calculated on the amount of the substance. The solutions were diluted 1/10 with a stabilizer solution consisting of 0.2% (w/w) PVP and 0.25 mM SDS in water. The critical mixing stage was rapid and instant:—The drug solution was rapidly injected into the stabilizer solution during ultrasonication. After 1/10 dilution in the aqeous solution, nanoparticles of about 110 nm were obtained. After 6 hours at room temperature, the particle sizes were unchanged.

Optionally DMA may be used instead of ethanol, Miglyol may be excluded and the dilution may be larger (1/20). Particles in the size range 100 to 300 nm may be obtained by different combinations.

### EXAMPLE 69

| | | |
|---|---|---|
| Compound B | 200 | μmol |
| PEG 400/ethanol/water 50/5/45 (w/w) % | to 1 | mL |

A formulation was prepared by dissolving Compound B in PEG 400/ethanol/water 50/5/45 (w/w) % followed by gently stirring. Formulations of B (at 0.5 mg/mL) in this vehicle are stable for at least 1 month at <−15° C.

### EXAMPLE 70

| | | |
|---|---|---|
| Compound B | 230 | μmol |
| PEG 400/ethanol/water 60/5/35 (w/w) % | to 1 | mL |

A formulation was prepared by dissolving Compound B in PEG 400/ethanol/60/5/35 (w/w) % followed by gently stirring.

### EXAMPLE 71

| | | |
|---|---|---|
| Compound B | 50 | mg |
| HPMC (15000 Cps) | 5 | mg |
| Solutol HS15 | 20 | mg |
| Water | to 1 | mL |

The HPMC was suspended in hot water and melted solutol was added during vigourous stirring. This solution was chilled and Compound B was added under vigourous stirring to form a well dispersed suspension.

### EXAMPLE 72

| | | |
|---|---|---|
| Compound E (as acetate salt) | 39 | μmol |
| 9 mg/ml NaCl | to 1 | mL |

A formulation was prepared by dissolving Compound E in 9 mg/ml NaCl by gently stirring. The pH obtained in this formulation is 8-9.

### EXAMPLE 73

| | | |
|---|---|---|
| Compound C | 400 | μmol |
| PEG 400/ethanol/water 50/5/45 (w/w) % | to 1 | mL |

A formulation was prepared by dissolving Compound C in PEG 400/ethanol/water 50/5/45 (w/w) % followed by gently stirring. Formulations of C (at 0.5 mg/mL) in this vehicle are stable for at least 1 month at room temperature and below.

### EXAMPLE 74

| | | |
|---|---|---|
| Compound C | 16 | μmol |
| Hydroxypropyl-β-cyclodextrin/water 20/80 (w/w) % | to 1 | mL |

A formulation was prepared by dissolving Compound C in Hydroxypropyl-β-cyclodextrin/water 20/80 (w/w) % followed by gently stirring. Formulations of C in this vehicle are stable for at least 2 weeks at <8° C.

### EXAMPLE 75

| | | |
|---|---|---|
| Compound F (as trifluoroacetate salt) | 38 | μmol |
| 9 mg/ml NaCl | to 1 | mL |

A formulation was prepared by dissolving Compound F in 9 mg/ml NaCl by gently stirring. The pH obtained in this formulation is 3-4. Formulations of F in this vehicle are stable for at least 2 weeks at at room temperature and below.

### EXAMPLE 76

A tablet was prepared according to the general method of Example 44.

| | Weight | Amount |
|---|---|---|
| Besylate salt of Compound A | 66 mg | 17% |
| Polyvinyl pyrrolidone K90 | 9 mg | 2% |
| Mannitol | 29 mg | 7% |
| Microcrystalline cellulose | 256 mg | 65% |
| Sodium starch glycolate | 29 mg | 7% |
| Sodium stearyl fumarate | 4 mg | 1% |

### Release Data

Measured according to the general method of Example 44 but using 500 ml of media and 75 rpm.

| | % released in |
|---|---|
| Time (min) | buffer pH 6.8 |
| 0 | 0 |
| 5 | 90 |
| 10 | 94 |
| 15 | 96 |
| 20 | 96 |
| 30 | 98 |
| 45 | 98 |
| 60 | 100 |

### EXAMPLE 77

A tablet is prepared according to the general method of Example 44.

| | Weight | Amount |
|---|---|---|
| Besylate salt of Compound A | 200 mg | 40% |
| Polyvinyl pyrrolidone K30 | 10 mg | 2% |
| Lactose | 200 mg | 40% |
| Microcrystalline cellulose | 70 mg | 14% |
| Polyvinylpolypyrrolidone CL | 15 mg | 3% |
| Magnesium stearate | 5 mg | 1% |

Other formulations in which the quantity of the besylate salt of Compound A is in the range 50-300 mg may be prepared; the ratio of other components being similar to those in Example 77.

### EXAMPLE 78

A tablet is prepared according to the general method of Example 44.

| | Weight | Amount |
|---|---|---|
| Hemi-Naphthalene 1,5-disulphonic | 48 mg | 17% |
| acid salt of Compound B | | |
| Polyvinyl pyrrolidone K90 | 8 mg | 3% |
| Mannitol | 21 mg | 7% |
| Microcrystalline cellulose | 187 mg | 65% |
| Sodium starch glycolate | 21 mg | 7% |
| Sodium stearyl fumarate | 3 mg | 1% |

Other formulations in which 100 mg or 200 mg of the hemi-naphthalene 1,5-disulphonic acid salt of Compound B is used may also be prepared; the ratio of other components being similar to those in Example 78.

Particular aspects of the invention are provided as follows:—
1. An immediate release pharmaceutical formulation comprising, as active ingredient, a compound of formula (I): 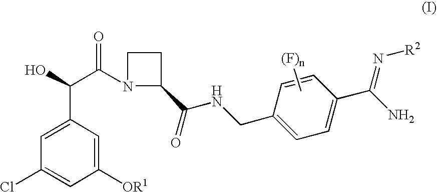
   wherein
   R¹ represents C₁₋₂ alkyl substituted by one or more fluoro substituents;
   R² represents hydrogen, hydroxy, methoxy or ethoxy; and
   n represents 0, 1 or 2;
   or a pharmaceutically acceptable salt thereof; and a pharmaceutically acceptable diluent or carrier;
   provided that the formulation does not solely contain:
      a solution of one active ingredient and water;
      a solution of one active ingredient and dimethylsulphoxide; or,
      a solution of one active ingredient in a mixture of ethanol:PEG 660 12-hydroxy stearate:water 5:5:90.
2. An immediate release pharmaceutical formulation as described in aspect 1 wherein the active ingredient is:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe);
Ph(3-Cl)(5-OCH₂CH₂F)—(R)CH(OH)C(O)—(S)Aze-Pab(OMe);
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(OH);
Ph(3-Cl)(5-OCHF₂)—(R)CH(OH)C(O)—(S)Aze-Pab(2,6-diF);
Ph(3-Cl)(5-OCHF₂)—(R)CH(OH)C(O)—(S)Aze-Pab(2,6-diF)(OH);
Ph(3-Cl)(5-OCH₂CH₂F)—(R)CH(OH)C(O)—(S)Aze-Pab; or,
Ph(3-Cl)(5-OCH₂CH₂F)—(R)CH(OH)C(O)—(S)Aze-Pab(OH).
3. A solid immediate release pharmaceutical formulation as described in aspect 1 wherein the active ingredient is:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe); or,
Ph(3-Cl)(5-OCH₂CH₂F)—(R)CH(OH)C(O)—(S)Aze-Pab(OMe),
or a pharmaceutically acceptable salt thereof.
4. A solid immediate release pharmaceutical formulation as described in aspect 1 wherein the active ingredient is Ph(3-Cl)(5-OCHF₂)—(R)CH(OH)C(O)—(S)Aze-Pab(OMe) or a C₁₋₆ alkanesulfonic acid or an optionally substituted arylsulfonic acid salt thereof.
5. An injectable immediate release pharmaceutical formulation as described in aspect 1 wherein the active ingredient is:
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(2,6-diF); or
Ph(3-Cl)(5-OCH₂CH₂F)—(R)CH(OH)C(O)—(S)Aze-Pab.
6. The use of a formulation as described in aspect 1 as a medicament.
7. The use of a formulation as described in aspect 1 in the manufacture of a medicament for the treatment of a cardiovascular disorder.
8. A method of treating a cardiovascular disorder in a patient suffering from, or at risk of, said disorder, which comprises administering to the patient a therapeutically effective amount of a pharmaceutical formulation as described in aspect 1.
9. A process for making an immediate release formulation as described in aspect 1.
10. The compound Ph(3-Cl)(5-OCHF₂)—(R)CH(OH)C(O)—(S)Aze-Pab(2,6-diF)(OH).

Also provided is a formulation obtainable by any of the Methods and/or Examples described herein.

1. An immediate release pharmaceutical formulation comprising, as an active ingredient, a compound of formula (I): 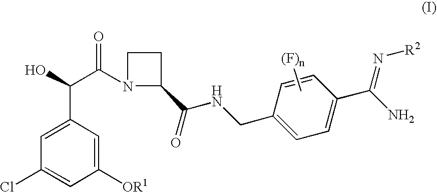
wherein
R¹ is C₁₋₂ alkyl substituted with one or more fluoro substituents;
R² is hydrogen, hydroxy, methoxy or ethoxy; and
n is 0 or 2;
or a pharmaceutically acceptable salt thereof; and
a pharmaceutically acceptable diluent or carrier; provided that when the active ingredient is other than in the form of a salt, the formulation does not solely contain:
a solution of one active ingredient and water;
a solution of one active ingredient and dimethylsulphoxide; or
a solution of one active ingredient in a mixture of ethanol:PEG 660 12-hydroxy stearate:water 5:5:90.

2. An immediate release pharmaceutical formulation as claimed in claim 1, comprising an acid addition salt of a compound of formula (I) and a pharmaceutically acceptable diluent or carrier.

3. An immediate release pharmaceutical formulation as claimed in claim 1, wherein the active ingredient is:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe);
Ph(3-Cl)(5-OCH₂CH₂F)—(R)CH(OH)C(O)—(S)Aze-Pab(OMe);
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(OH);
Ph(3-Cl)(5-OCHF₂)—(R)CH(OH)C(O)—(S)Aze-Pab(2,6-diF);
Ph(3-Cl)(5-OCHF₂)—(R)CH(OH)C(O)—(S)Aze-Pab(2,6-diF)(OH);
Ph(3-Cl)(5-OCH₂CH₂F)—(R)CH(OH)C(O)—(S)Aze-Pab; or
Ph(3-Cl)(5-OCH₂CH₂F)—(R)CH(OH)C(O)—(S)Aze-Pab(OH).

4. A formulation as claimed in claim 1, wherein the active ingredient is a crystalline salt of:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe); or
Ph(3-Cl)(5-OCH₂CH₂F)—(R)CH(OH)C(O)—(S)Aze-Pab(OMe).

5. A formulation as claimed in claim 1, wherein the active ingredient is an ethanesulfonic acid, n-propanesulfonic acid, benzenesulfonic acid, 1,5-naphthalenedisulfonic acid, or n-butanesulfonic acid addition salt of Ph(3-Cl)(5-OCHF₂)—(R)CH(OH)C(O)—(S)Aze-Pab(OMe) or Ph(3-Cl)(5-OCHF₂)—(R)CH(OH)C(O)—(S)Aze-Pab(2,6-diF)(OMe).

6. A formulation as claimed in claim 1, wherein the active ingredient is Ph(3-Cl)(5-OCHF₂)—(R)CH(OH)C(O)—(S)Aze-Pab(OMe), benzene-sulfonic acid salt, characterised by an X-ray powder diffraction pattern characterised by peaks with d-values at 5.9, 4.73, 4.09, and 4.08 Å.

7. A formulation as claimed in claim 1, wherein the active ingredient is Ph(3-Cl)(5-OCHF₂)—(R)CH(OH)C(O)—(S)Aze-Pab(2,6-diF)(OMe), hemi-1,5-naphthalenedisulfonic acid salt, characterised by an X-ray powder diffraction pattern characterised by peaks with d-values at 18.3, 9.1, 5.6, 5.5, 4.13, 4.02, 3.86, 3.69, and 3.63 Å.

8. A formulation as claimed in claim 1, wherein the composition is selected from a solid immediate release pharmaceutical formulation, an injectable immediate release pharmaceutical formulation, or a liquid immediate release oral pharmaceutical formulation.

9. A method for treating a patient suffering from, or at risk of developing a cardiovascular disorder, comprising administering to the patient a therapeutically effective amount of a pharmaceutical formulation of any one of claims 1 to 8.
